(19) United States
(12) Patent Application Publication
(10) Pub. No.: US 2026/0055474 A1
(43) Pub. Date: Feb. 26, 2026

(54) QUANTITATIVE TRAIT LOCUS ASSOCIATED WITH A FLOWER DENSITY TRAIT IN CANNABIS

(71) Applicant: Puregene AG, Zeiningen (CH)

(72) Inventors: Michael Edward Ruckle, Zeiningen (CH); Gavin Mager George, Zeiningen (CH); Claudio Cropano, Zeiningen (CH); Leron Katsir, Zeiningen (CH); Maximilian Moritz Vogt, Zeiningen (CH); Dániel Árpád Carrera, Zeiningen (CH); Michele Wyler, Zeiningen (CH); Eduard Bruderer, Zeiningen (CH); Marc Schmid, Zeiningen (CH); Mercedes Thieme, Zeiningen (CH)

(21) Appl. No.: 18/876,368

(22) PCT Filed: Jun. 21, 2023
(86) PCT No.: PCT/IB2023/056411

§ 371 Date: Dec. 18, 2024

(30) Foreign Application Priority Data
Jun. 21, 2022 (GB) 2209127.6

(51) Int. Cl. C12Q 1/6895; A01H 1/00; A01H 1/04; A01H 5/02; A01H 6/28

(52) U.S. Cl. CPC C12Q 1/6895; A01H 1/045; A01H 1/12; A01H 5/02; A01H 6/28; C12Q 2600/13; C12Q 2600/156

(57) ABSTRACT

The invention relates to methods of identifying and characterizing a Cannabis spp. plant comprising a quantitative trait locus (QTL) or a causal polymorphism associated with a flower density trait, and to Cannabis spp. plants having a flower density trait of interest comprising defined allelic states of polymorphisms defining the QTL or defined allelic states of causal polymorphisms provided herein. Also provided are Cannabis spp. plants with a flower density trait of interest comprising defined allelic states of polymorphisms and plants identified, characterized or produced by the methods described. Further provided are methods of marker assisted selection, genomic selection, marker assisted breeding, and genetic modification, for obtaining plants having a flower density trait of interest.

### BACKGROUND OF THE INVENTION

The invention relates to methods of identifying and characterizing a Cannabis spp. plant comprising a quantitative trait locus (QTL) associated with a flower density trait, and to Cannabis spp. plants having a flower density trait of interest comprising defined allelic states of the polymorphisms defining the QTL or the allelic states of causal polymorphisms provided herein. The invention further relates to Cannabis spp. plants with a flower density trait of interest identified, characterized, or produced by the methods described herein. The invention also relates to marker assisted selection and marker assisted breeding methods for obtaining plants having a flower density trait of interest. Also provided are methods of producing Cannabis spp. plants with the flower density trait of interest and plants produced by these methods, based on the allelic state of the QTLs, as well as genes responsible for controlling the flower density trait of interest.

Modern Cannabis is derived from the cross hybridization of three biotypes; Cannabis sativa L. ssp. indica, Cannabis sativa L. ssp. sativa, and Cannabis sativa L. ssp. ruderalis. Cannabis was divergently bred into two distinct, albeit tentative types, called Hemp and HRT (high-resin-type) Cannabis, respectively, which are used for different purposes. Hemp is primarily used for industrial purposes, for example in feed, food, seed, fiber, and oil production. Conversely, HRT Cannabis is largely cultivated and bred for high concentrations of the pharmacological constituents, cannabinoids, derived from resin in the trichomes. Biomass, including the leaf and stem, of Cannabis can also be an important source of cannabinoids.

Cannabis is the only species in the plant kingdom cultivated to produce phytocannabinoids. Phytocannabinoids are a class of terpenoid acting as antagonists and agonists of mammalian endocannabinoid receptors. The pharmacological action is derived from the ability of phytocannabinoids to disrupt and mimic endocannabinoids. Due to its psychoactive properties, one cannabinoid, delta-9-tetrahydrocannabinol (THC), the decarboxylation product of the plant-produced delta-9-tetrahydrocannabinolic acid (THCA), has received much attention in illegal or unregulated breeding programs, with modern HRT varieties having THC concentrations of 0.5% to 30%.

Cannabis is a dioecious plant, having male and female flowers produced on separate plants. In HRT Cannabis production, female plants are cultivated for their flowers, a source of high cannabinoid content. Cannabis flowers display a range of flower densities and sizes with some flowers being highly compact and others being highly diffuse. Flower size and flower density of Cannabis are important economic traits because they are major determinants of dry flower yield and cannabinoid content per gram. High yielding plants are important to producers needing to maximize yield per area under cultivation. Dense Cannabis flowers are visually more appealing and are preferred by consumers of resin type Cannabis. On the other hand, dense flowers may be more susceptible to fungal diseases like grey mould because of the humid microclimates of compact Cannabis flowers. In hemp-type Cannabis increased flower density can lead to improvements in total seed yield.

Understanding the genetic basis and molecular mechanisms of flower size and flower density are important to improve traits related to Cannabis flower and seed yield as well as flower quality.

In addition, flower density can also play an important role in the study of evolution of Cannabis varieties. It is generally accepted that hemp type Cannabis flowers are sparse in comparison to resin type Cannabis that has been selected for flower traits, including density. Even so, many Cannabis HRT varieties suffer from low flower density, having highly incompact buds.

Flower density is a quantitative trait and has a complex genetic architecture. Growth conditions, including temperature, light quality, nutrient availability, and planting density may also influence final flower density. The Cannabis sativa inflorescence is a compound raceme, on which secondary or higher order inflorescences develop from the primary inflorescence and bear flowers. The flowers are closely aggregated at the apex of short spike inflorescences. As the axis of primary inflorescence is elongated, secondary inflorescences are continuously generated laterally and bear flowers in succession. Arabidopsis thaliana inflorescence, for contrast, is a simple raceme, as the flowers are borne on the main stem of the primary inflorescence. In Cannabis, each female flower has an ovary with a style that ends in a pair of long slender feathery stigmas, a membranous perianth surrounds the ovary, and is enclosed in a bract. The bracts are modified leaves that are covered in stalked glandular trichomes that contain the highest concentrations of cannabinoid in the Cannabis plant. Flower density may relate to the stem elongation between upper or axillary inflorescences, the number of individual inflorescences per primary and secondary shoot, or the size of the flower organs, for example the bracts.

The genetic factors that control flower structure may be related to the genetic control of flower density. The interplay between the inflorescence meristem (IM), that gives rise to the floral meristem (FM), bracts, and additional IMs, and the FM are important to inflorescence architecture. Inflorescence architecture has been majorly studied in the simple raceme Arabidopsis where the genes TERMINAL FLOWER (TFL1), APETALA1 (AP1), UNUSUAL FLOWER ORGAN (UFO), and LEAFY (LFY) are major regulators of IM to FM transitions and the regulation of floral identity. It has previously not been possible to identify a comparable phenotype in Arabidopsis or other plant species from which to make a determination of the gene or gene family that may be involved in flower density in Cannabis. This is mainly due to the still limited research on flower architecture and development in crop species but also because of the varied inflorescence architecture when compared to the Cannabis compound raceme structure. In mungbean, which also has a compound raceme inflorescence, a gene that is a homolog of Arabidopsis ABNORMAL SHOOT 2 gene was proposed to play a role in mungbean plants found with a simple raceme inflorescence architecture. It is not clear that this phenotypic shift in flower architecture is analogous to the diversity of floral density observed in Cannabis. In Cannabis, flower density has not been studied and no genes or genetic regions have been identified that regulate flower density or compactness.

In the present invention, a novel approach to characterizing the density of Cannabis flowers was coupled to the genetic diversity and population structure of 11 F2 populations in order to conduct a genome wide association (GWA) to determine the genetic factors influencing flower density in Cannabis.

### SUMMARY OF THE INVENTION

The invention relates to methods of identifying and characterizing a Cannabis spp. plant comprising a quantitative trait locus (QTL) or a causal polymorphism associated with a flower density trait, and to Cannabis spp. plants having a flower density trait of interest comprising defined allelic states of polymorphisms defining the QTL. Also provided are methods of producing a Cannabis spp. plant having a flower density trait of interest and plants with a flower density trait of interest identified or produced by the methods described herein. The invention further relates to marker assisted selection, genomic selection, and marker assisted breeding methods for obtaining plants having a flower density trait of interest, as well as to methods of producing Cannabis spp. plants with a flower density QTL based on defined allelic states of causal polymorphisms within the QTL or polymorphisms linked thereto and to plants comprising the flower density QTL. Further provided are isolated nucleic acids comprising a QTL that controls a flower density trait, as well as an isolated gene, comprising specific causal polymorphisms that control the flower density trait in Cannabis spp.

According to a first aspect of the present invention there is provided for a method for characterizing a Cannabis spp. plant with respect to a flower density trait, the method comprising the steps of: (i) genotyping at least one plant with respect to a flower density QTL by detecting: (a) one or more polymorphisms associated with the flower density trait as defined in Table 2 or 3; and/or (b) a polymorphism causal for the flower density trait selected from a A/G SNP at position 685 of SEQ ID NO:XX and a T/C SNP at position 1271 of SEQ ID NO:45; and (ii) characterizing the plant with respect to the flower density QTL as having an increased flower density QTL, a decreased flower density QTL or an intermediate flower density QTL, based on the genotype at the polymorphism.

In a first embodiment of the method for characterizing a Cannabis spp. plant with respect to a flower density trait, the polymorphism may be selected from the group consisting of “common_563”, “GBScompat_rare_14”, “common_573”, “GBScompat_common_102”, “rare_66”, “common_583”, and combinations thereof, as defined in Table 2 or 3. Further, the molecular marker “common_563” has been shown to have particularly high predictive value for the flower density QTL and trait. In some embodiments, the polymorphism is selected from the A/G SNP at position 685 of SEQ ID NO:45 and the T/C SNP at position 1271 of SEQ ID NO:45, or both. In some embodiments, with reference to the polymorphism at position 685 of SEQ ID NO:45, a plant having a homozygous AA genotype at this position has a propensity for an increased flower density trait, a plant having a heterozygous AG genotype at this position has a propensity for an intermediate flower density trait, and a plant having a homozygous GG genotype at this position has a propensity for a decreased flower density trait. In some embodiments, with respect to the causal polymorphism at position 1271 of SEQ ID NO:45, a plant having a homozygous TT genotype at this position has a propensity for an increased flower density trait, a plant having the heterozygous TC genotype at this position has a propensity for an intermediate flower density trait, and a plant having a homozygous CC genotype at this position has a propensity for a decreased flower density trait. It will further be appreciated by those of skill in the art, that although the polymorphisms at position 685 of SEQ ID NO:45 and at position 1271 of SEQ ID NO:45 have been identified as causal polymorphisms, all of the polymorphisms provided in Table 2 or 3 have predictive value in characterizing the Cannabis spp. plant for the flower density trait.

In a second embodiment of the method for characterizing a Cannabis spp. plant with respect to a flower density trait, the genotyping may be performed by any PCR-based detection method using molecular markers, by sequencing of PCR products containing the one or more polymorphisms, by targeted resequencing, by whole genome sequencing, or by restriction-based methods, for detecting the one or more polymorphisms. Several such methods are known in the art, and those of skill in the art will appreciate that any method of detection that is capable of distinguishing allelic states of polymorphisms may be employed in the methods of the invention.

In a third embodiment of the method for characterizing a Cannabis spp. plant with respect to a flower density trait, the molecular markers may be for detecting polymorphisms at regular intervals within the flower density QTL such that recombination can be excluded. In an alternative embodiment, the molecular markers may be for detecting polymorphisms at regular intervals within the flower density QTL such that recombination can be quantified to estimate linkage disequilibrium between a particular polymorphism and the flower density phenotype. It will be appreciated by those of skill in the art that several possible markers may be designed for detecting the polymorphisms. For example, molecular markers may be for detecting polymorphisms such that recombination events can be detected to a resolution of 10′000 or 100′000 or 500′000 base pairs within the QTL. In one embodiment, the molecular markers may be designed based on a context sequence for the polymorphism provided in Table 3 or may be selected from the primer pairs as defined in Table 4.

According to a fourth embodiment of the method for characterizing a Cannabis spp. plant with respect to a flower density trait, the flower density QTL is a quantitative trait locus having a sequence that corresponds to nucleotides 102037098 to 104628858 of NC_044370.1 of the CS10 genome and is defined by one or more polymorphisms associated with flower density as defined in Table 2 or 3, or a genetic marker linked to the QTL. In some embodiments, the flower density QTL has a sequence that is substantially identical, such as 99% identical, 98% identical, 97% identical, 96% identical, or 95% identical, to the sequence represented by nucleotides 102037098 to 104628858 of NC_044370.1 of the CS10 genome and is defined by one or more polymorphisms associated with flower density as defined in Table 2 or 3, or a genetic marker linked to the QTL.

According to a second aspect of the present invention, there is provided for a method of producing a Cannabis spp. plant having a flower density trait of interest, the method comprising the steps of: (i) providing a donor parent plant having in its genome a flower density QTL characterized by: (a) one or more polymorphisms associated with the flower density trait of interest as defined Table 2 or 3; and/or (b) a polymorphism causal for the flower density trait of interest selected from a A/G SNP at position 685 of SEQ ID NO:45 and a T/C SNP at position 1271 of SEQ ID NO:45; (ii) crossing the donor parent plant having the flower density QTL with at least one recipient parent plant to obtain a progeny population of Cannabis plants; (iii) screening the progeny population of Cannabis plants for the presence of the flower density QTL; and (iv) selecting one or more progeny plants having the flower density QTL, wherein the mature plant displays the flower density trait of interest. The flower density trait of interest may be an increased flower density trait, a decreased flower density trait. In this way, the trait can be modulated using the QTL and polymorphisms of the invention.

In a first embodiment of the method of producing a Cannabis spp. plant having a flower density trait of interest, the method may further comprise the steps of: (v) crossing the one or more progeny plants with the donor recipient plant; or (vi) selfing the one or more progeny plants.

According to a second embodiment of the method of producing a Cannabis spp. plant having a flower density trait of interest, the screening may comprise genotyping at least one plant from the progeny population with respect to a flower density QTL by detecting one or more polymorphisms associated with the flower density trait of interest as defined Table 2 or 3; and/or the polymorphism causal for the flower density trait of interest. In some embodiments, the progeny population of Cannabis plants contains a minimum of 100, or 500, or 1000, or 10000 plants.

In a third embodiment of the method of producing a Cannabis spp. plant having a flower density trait of interest, the method may further comprise a step of genotyping the donor parent plant with respect to a flower density QTL by detecting one or more polymorphisms associated with flower density trait of interest as defined Table 2 or 3; and/or the polymorphism causal for the flower density trait of interest, preferably prior to step (i).

According to a fourth embodiment of the method of producing a Cannabis spp. plant having a flower density trait of interest, the genotyping may be performed by a PCR-based detection using molecular markers, by sequencing of PCR products containing the one or more polymorphisms, by targeted resequencing, by whole genome sequencing, or by restriction-based methods, for detecting the one or more polymorphisms. Several such methods are known in the art, and those of skill in the art will appreciate that any method of detection that is capable of distinguishing allelic states of polymorphisms may be employed in the methods of the invention.

In a fifth embodiment of the method of producing a Cannabis spp. plant having a flower density trait of interest, the molecular markers may be for detecting polymorphisms at regular intervals within the flower density QTL such that recombination can be excluded. In an alternative embodiment, the molecular markers may be for detecting polymorphisms at regular intervals within the flower density QTL such that recombination can be quantified to estimate linkage disequilibrium between a particular polymorphism and the flower density phenotype. For example, molecular markers may be for detecting polymorphisms such that recombination events can be detected to a resolution of 10′000 or 100′000 or 500′000 base pairs within the QTL. It will be appreciated by those of skill in the art that several possible markers may be designed for detecting the polymorphisms. In one embodiment, the molecular markers may be designed based on a context sequence for the polymorphism provided in Table 3 or may be selected from the primer pairs as defined in Table 4.

According to a further embodiment of the method of producing a Cannabis spp. plant having a flower density trait of interest, the flower density QTL may an increased flower density QTL associated with increased flower density, a decreased flower density QTL associated with decreased flower density, or an intermediate flower density QTL associated with intermediate flower density, as defined in Table 2 or 3. Of particular use in producing a Cannabis spp. plant having a flower density trait of interest, are the polymorphisms selected from the group consisting of “common_563”, “GBScompat_rare_14”, “common_573”, “GBScompat_common_102”, “rare_66”, and “common_583”, as defined in Table 2 or 3. Further, the molecular marker “common_563” has been shown to have particularly high predictive value for the flower density QTL and trait. In some embodiments, the polymorphism is selected from the A/G SNP at position 685 of SEQ ID NO:45 and the T/C SNP at position 1271 of SEQ ID NO:45, or both. In some embodiments, with reference to the polymorphism at position 685 of SEQ ID NO:45, a plant having a homozygous AA genotype at this position has a propensity for an increased flower density trait, a plant having a heterozygous AG genotype at this position has a propensity for an intermediate flower density trait, and a plant having a homozygous GG genotype at this position has a propensity for a decreased flower density trait. In some embodiments, with respect to the causal polymorphism at position 1271 of SEQ ID NO:45, a plant having a homozygous TT genotype at this position has a propensity for an increased flower density trait, a plant having the heterozygous TC genotype at this position has a propensity for an intermediate flower density trait, and a plant having a homozygous CC genotype at this position has a propensity for a decreased flower density trait. It will further be appreciated by those of skill in the art, that although the polymorphisms at position 685 of SEQ ID NO:45 and at position 1271 of SEQ ID NO:45 have been identified as causal polymorphisms, all of the polymorphisms provided in Table 2 or 3 have predictive value in characterizing the Cannabis spp. plant for the flower density trait.

In one embodiment, the flower density trait of interest is an increased flower density trait and the flower density QTL is an increased flower density QTL, or the flower density trait of interest is an intermediate flower density trait and the flower density QTL is an intermediate flower density QTL, or the flower density trait of interest is a decreased flower density trait and the flower density QTL is a decreased flower density QTL.

In a further embodiment of the method for producing a Cannabis spp. plant comprising a flower density trait of interest, the flower density QTL is a quantitative trait locus having a sequence that corresponds to nucleotides 102037098 to 104628858 of NC_044370.1 of the CS10 genome and is defined by one or more polymorphisms associated with flower density as defined in Table 2 or 3, or a genetic marker linked to the QTL. In some embodiments, the flower density QTL has a sequence that is substantially identical, such as 99% identical, 98% identical, 97% identical, 96% identical, or 95% identical, to the sequence represented by nucleotides 102037098 to 104628858 of NC_044370.1 of the CS10 genome and is defined by one or more polymorphisms associated with flower density as defined in Table 2 or 3, or a genetic marker linked to the QTL.

According to a third aspect of the present invention there is provided for a method of producing a Cannabis spp. plant comprising a flower density trait of interest, wherein the method comprises introducing into a Cannabis spp. plant a flower density QTL: (a) characterized by one or more polymorphisms associated with the flower density trait of interest as defined in Table 2 or 3, wherein said flower density QTL is associated with the flower density trait of interest in the plant; and/or (b) comprising a polymorphism causal for the flower density trait of interest selected from a A/G SNP at position 685 of SEQ ID NO:45 and a T/C SNP at position 1271 of SEQ ID NO: 45, or both such polymorphisms. In one embodiment, introducing the flower density QTL may comprise crossing a donor parent plant having the flower density QTL with a recipient parent plant. In an alternative embodiment, introducing the flower density QTL may comprise genetically modifying the Cannabis spp. plant. Several methods of genetic modification are known to those of skill in the art, including targeted mutagenesis, genome editing, and gene transfer. For example, one or more of the polymorphisms associated with the flower density trait of interest as defined in Table 2 or 3 herein may be introduced into a plant by mutagenesis and/or gene editing. In particular, the Cannabis spp. plant may be genetically modified by targeted mutagenesis of a nucleotide corresponding to the position 685 of SEQ ID NO:45, position 1271 of SEQ ID NO:45, or both. Methods of genetically modifying a plant may be selected from the group consisting of CRISPR-Cas9 targeted gene editing, heterologous gene expression using various expression cassettes; TILLING, and non-targeted chemical mutagenesis using e.g. EMS. Alternatively, a Cannabis spp. plant may be transformed with a cassette containing the QTL associated with the flower density trait of interest or a part thereof, via any transformation method known in the art.

In some embodiments of the method of producing a Cannabis spp. plant comprising a flower density trait of interest, the method comprises introducing into a Cannabis spp. plant a flower density QTL having a sequence that corresponds to nucleotides 102037098 to 104628858 of NC_044370.1 of the CS10 genome and which is defined by one or more polymorphisms associated with flower density as defined in Table 2 or 3, or a genetic marker linked to the QTL. In some embodiments, the flower density QTL has a sequence that is substantially identical, such as 99% identical, 98% identical, 97% identical, 96% identical, or 95% identical, to the sequence represented by nucleotides 102037098 to 104628858 of NC_044370.1 of the CS10 genome and is defined by one or more polymorphisms associated with flower density as defined in Table 2 or 3, or a genetic marker linked to the QTL.

According to a fourth aspect of the present invention there is provided for a Cannabis spp. plant characterized according to the method for characterizing a Cannabis spp. plant with respect to a flower density trait as described herein, or produced according to the method of producing a Cannabis spp. plant that has a flower density trait of interest as described herein. In some embodiments, the Cannabis spp. plant thus characterized or produced is not exclusively obtained by means of an essentially biological process.

According to a further aspect of the present invention there is provided for a Cannabis spp. plant comprising a flower density QTL, wherein the flower density QTL is: (a) characterized by one or more polymorphisms associated with a flower density trait of interest as defined in Table 2 or 3, wherein said flower density QTL is associated with the flower density trait of interest in the plant; and/or (b) comprising a polymorphism causal for the flower density trait of interest selected from a A/G SNP at position 685 of SEQ ID NO:45 and a T/C SNP at position 1271 of SEQ ID NO: 45, provided that the plant is not exclusively obtained by means of an essentially biological process.

According to another aspect of the present invention there is provided for an isolated nucleic acid molecule comprising a quantitative trait locus that controls a flower density trait in Cannabis spp., wherein the quantitative trait locus has a nucleic acid sequence that corresponds to nucleotides 102037098 to 104628858 of NC_044370.1 with reference to the CS10 genome and is defined by one or more polymorphisms associated with flower density as defined in Table 2 or 3, or a genetic marker linked to the QTL. In one embodiment, the invention further includes a genomic region defined by markers linked to the flower density QTL defined herein.

In a further aspect there is provided for an isolated gene that controls a flower density trait in a Cannabis spp. plant, wherein the gene is selected from the group consisting of the genes as defined in Table 7 with reference to the CS10 genome. The isolated gene may be selected from the group consisting of a gene having the gene identity number LOC115701253, or LOC115701243, and encoding a BEL1-like protein; a gene having the gene identity number LOC115703213 and encoding a Cytokinin Dehydrogenase; and a gene having the gene identity number LOC115701693, LOC115703227, or LOC115701761, and encoding an auxin-responsive factor AUX/IAA-like protein.

In one embodiment, the gene is a gene having the gene identity number LOC115703213 and encoding a Cytokinin Dehydrogenase, wherein the gene has a single nucleotide polymorphism “pos_1093_A” at position 103015494, as defined in Table 3.

According to a further aspect of the present invention there is provided for an isolated gene that controls a flower density trait in a Cannabis spp. plant, wherein the gene has the NCBI gene identity number LOC115702276 and encodes a homolog of a transcriptional corepressor LEUNIG isoform X1-X4 protein from Arabidopsis. In one embodiment of the isolated gene that controls a flower density trait, the gene is substantially identical to SEQ ID NO:45, such as 99% identical, 98% identical, 97% identical, 96% identical, or 95% identical, to the sequence of SEQ ID NO: 45. According to a further embodiment of the isolated gene that controls a flower density trait in a Cannabis spp. plant, the gene comprises one or both polymorphisms causal for the flower density trait selected from a A/G SNP at position 685 of SEQ ID NO:45 and a T/C SNP at position 1271 of SEQ ID NO:45.

### BRIEF DESCRIPTION OF THE FIGURES

Non-limiting embodiments of the invention will now be described by way of example only and with reference to the following figures:

FIG. 1: Flower density measurements represented in a box plot for each F2 population. The box plot shows density as represented by flower weight/area on the Y-axis. On the X-axis are the individual F2 populations. Outliers are represented as single black dots. Error bars are the standard deviation. Central black bars in the middle of the box indicate the mean density for the population.

FIG. 2: A GWA of flower density in Cannabis in a mixed F2 population.

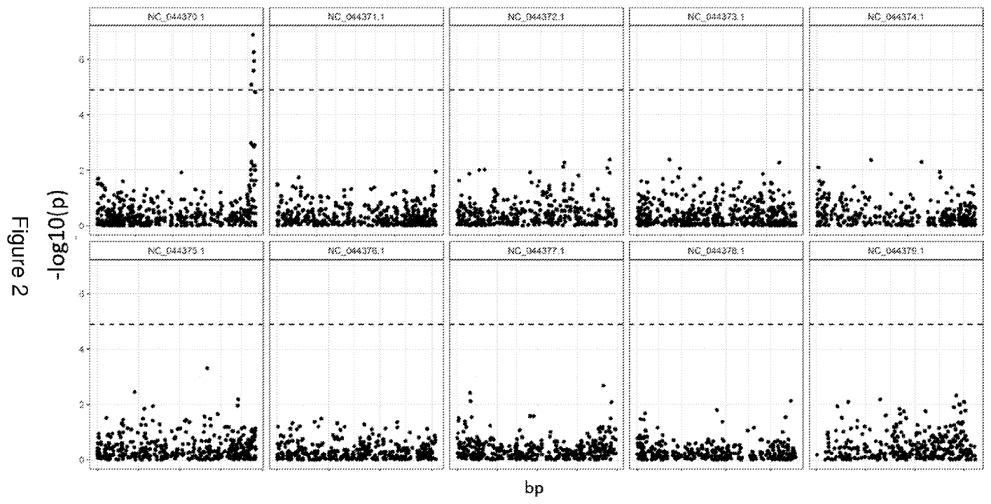

FIG. 3: A highly conserved region in Cannabis CS20 CKXs: Alignment of Cannabis CS10 CKXs in selected region of interest. Asterisks indicate highly conserved amino acids. Underlined asterisk is the conserved G365 identified in XP_030486587.1.

### SEQUENCES

The nucleic acid and amino acid sequences listed herein and in any accompanying sequence listing are shown using standard letter abbreviations for nucleotide bases, and the standard one or three letter abbreviations for amino acids. It will be understood by those of skill in the art that only one strand of each nucleic acid sequence is shown, but that the complementary strand is included by any reference to the displayed strand.

### DETAILED DESCRIPTION OF THE INVENTION

The present invention will now be described more fully hereinafter with reference to the accompanying drawings, in which some, but not all embodiments of the invention are shown.

The invention as described should not be limited to the specific embodiments disclosed and modifications and other embodiments are intended to be included within the scope of the invention. Although specific terms are employed herein, they are used in a generic and descriptive sense only and not for purposes of limitation.

As used throughout this specification and in the claims, which follow, the singular forms “a”, “an” and “the” include the plural form, unless the context clearly indicates otherwise.

The terminology and phraseology used herein is for the purpose of description and should not be regarded as limiting. The use of the terms “comprising”, “containing”, “having” and “including” and variations thereof used herein, are meant to encompass the items listed thereafter and equivalents thereof as well as additional items. It is, however, contemplated as a specific embodiment of the present disclosure that the term “comprising” encompasses the possibility of no further members being present, i.e., for the purpose of such an embodiment “comprising” is to be understood as having the meaning of “consisting of”.

Methods are provided herein for characterizing, identifying, breeding and obtaining plants having an increased flower density trait prior to the plant displaying the flower density phenotypically, using a molecular marker detection technique. Such molecular markers may be employed in methods of selection and breeding to obtain plants with a flower density trait of interest. The inventors were able to use genome wide association (GWA) to identify a single QTL linked to flower density. They were further able to map the flower density trait to candidate genes, including a candidate gene containing causal SNPs that regulates the flower density trait. This finding provides for the improvement of methods for producing plants displaying differing degrees of flower density. In addition, this finding provides a method of prescreening a population for the flower density trait prior to the appearance of the trait.

One QTL for flower density was identified and confirmed in the mixed populations of 11 F2 populations tested.

Table 2 herein provides several single nucleotide polymorphisms (SNPs) which define the QTL associated with the flower density trait and which can be used for characterizing a plant with respect to the flower density trait. Context sequences for the SNPs are provided in Table 3 herein. In some embodiments one or more of the identified SNPs can be used to incorporate a haplotype of the flower density trait from a donor plant, containing the QTL associated with the trait, into a recipient plant. For example, the incorporation of the increased flower density phenotype may be performed by crossing a donor parent plant to a recipient parent plant to produce plants containing a haploid genome from both parents. Recombination of these genomes provides F1 progeny where each haploid complement of chromosomes, of the diploid genome, is comprised of genetic material from both parents.

In some embodiments, methods of identifying a QTL that is characterized by a haplotype comprising of a series of polymorphisms in linkage disequilibrium are provided. The QTL displays limited frequency of recombination within the QTL. Preferably the polymorphisms are selected from Table 2 or 3 herein, representing the flower density QTL. Molecular markers may be designed for use in detecting the presence of the polymorphisms and thus the QTL. Further, the identified QTL polymorphisms and the associated molecular markers may be used in a Cannabis breeding program to predict or modulate the flower density of plants in a breeding population and can be used to produce Cannabis plants that either display the increased flower density trait or display the decreased- or intermediate-flower density trait, or which have an increased or reduced propensity for the trait compared to the plants from which they are derived.

As used herein, reference to a “flower density” or a “flower density trait” refers the relationship between flower weight (g) and flower area (cm²), flower weight/area (g/cm²) for non-pollinated and pollinated flowers. Flower density may relate to the stem elongation between upper or axillary inflorescences, the number of individual inflorescences per primary and secondary shoot, or the size of the flower organs, for example the bracts.

A “flower density trait of interest” refers to the state of the plant with respect to the flower density trait and includes an increased flower density trait, an intermediate flower density trait and a decreased flower density trait.

As used herein, reference to a plant or variety with an “increased flower density trait” refers to a plant or variety having a propensity for increased flower density compared to plants from which it is derived. In some cases, a plant or variety with an increased flower density trait has a propensity for increased flower density in comparison to the mean flower density of plants from a population from which the plant or variety was derived.

As used herein, reference to a plant or variety with a “decreased flower density trait” refers to a plant or variety having a propensity for decreased flower density compared to plants from which it is derived. In some cases, a plant or variety with an decreased flower density trait has a propensity for decreased flower density in comparison to the mean flower density of plants from a population from which the plant or variety was derived.

As used herein, reference to a plant or variety with an “intermediate flower density trait” refers to a plant or variety having a propensity for intermediate flower density compared to plants from which it is derived. In some cases, a plant or variety with an intermediate flower density trait has a propensity for intermediate or average flower density in comparison to the mean flower density of plants from a population from which the plant or variety was derived.

The time of harvest is defined with respect to the maturity of the flower, where approximately greater than 50% of the pistils have turned brown in appearance. Alternatively, the time of harvest can also be determined by initiation of flowering for hemp-type Cannabis or by other agronomic criteria common in the art.

It is a particular aim of the present invention to identify and characterize a plant for the flower density trait of interest early in the plant lifecycle, particularly prior to the plant displaying the flower density trait, for example to ensure the flower density trait of interest is present in the breeding population early on. This can be achieved by genotyping the plant using molecular markers for detecting a QTL associated with the flower density trait of interest prior to the time of harvest.

As used herein a “quantitative trait locus” or “QTL” is a polymorphic genetic locus with at least two alleles that differentially affect the expression of a continuously varying phenotypic trait when present in a plant or organism which is characterised by a series of polymorphisms in linkage disequilibrium with each other.

As used herein, the term “flower density QTL” or “flower density quantitative trait locus” refers to a quantitative trait locus comprising part, or all, of the QTL characterized by the polymorphisms having an allelic state associated with the flower density trait of interest as described Table 2 or 3. The flower density QTL may be an increased flower density QTL, a decreased flower density QTL, or an intermediate flower density QTL as defined herein.

In some cases, it is desirable to obtain a plant displaying an increased flower density trait. In other embodiments, it is desirable to obtain a plant displaying a decreased or intermediate flower density trait. Thus, it is an objective of the invention to provide for Cannabis plants having an increased flower density QTL, a decreased flower density QTL, or an intermediate flower density QTL as described herein.

As used herein, the term “increased flower density QTL” or “increased flower density quantitative trait locus” refers to a quantitative trait locus comprising one or more polymorphisms having an allelic state associated with, or conferring, an increased flower density trait as described or defined in Table 2 or 3.

As used herein, the term “decreased flower density QTL” or “decreased flower density quantitative trait locus” refers to a quantitative trait locus comprising one or more polymorphisms having an allelic state associated with, or conferring, a decreased flower density trait as described or defined in Table 2 or 3.

As used herein, the term “intermediate flower density QTL” or “intermediate flower density quantitative trait locus” refers to a quantitative trait locus comprising one or more polymorphisms having an allelic state associated with, or conferring, an intermediate flower density trait as described or defined in Table 2 or 3.

As used herein, “haplotypes” refer to patterns or clusters of alleles or single nucleotide polymorphisms that are in linkage disequilibrium and therefore inherited together from a single parent. The term “linkage disequilibrium” refers to a non-random segregation of genetic loci or markers. Markers or genetic loci that show linkage disequilibrium are considered linked.

As used herein, the term “flower density haplotype” refers to the subset of the polymorphisms contained within the flower density QTL which exist on a single haploid genome complement of the diploid genome, and which are in linkage disequilibrium with the flower density trait.

As used herein, the term “increased flower density haplotype” refers to the subset of the polymorphisms contained within the increased flower density QTL which exist on a single haploid genome complement of the diploid genome, and which are in linkage disequilibrium with the increased flower density trait.

As used herein, the term “decreased flower density haplotype” refers to the subset of the polymorphisms contained within the decreased flower density QTL which exist on a single haploid genome complement of the diploid genome, and which are in linkage disequilibrium with the decreased flower density trait.

As used herein, the term “donor parent plant” refers to a plant having a flower density haplotype, or one or more flower density alleles, associated with the flower density trait of interest.

As used herein, the term “recipient parent plant” refers to a plant having a flower density haplotype, or one or more flower density alleles, not associated with the flower density trait of interest.

The term “flower density allele” refers to the haplotype allele state within the QTL that confers, or contributes to, the flower density trait of interest, or alternatively, is an allele that allows the identification of plants with the flower density trait of interest, and that can be included in a breeding program, particularly to select for the flower density trait of interest (“marker assisted breeding”, “marker assisted selection”, or “genomic selection”).

The term “crossed” or “cross” means the fusion of gametes via pollination to produce progeny (e.g., cells, seeds or plants). The term encompasses both sexual crosses (the pollination of one plant by another) and selfing (self-pollination, e.g., when the pollen and ovule are from the same, or genetically identical plant). The term “crossing” refers to the act of fusing gametes via pollination to produce progeny.

The term “GWAS” or “Genome wide association study” or “GWA” or “Genome wide association” as used herein refers to an observational study of a genome-wide set of genetic variants or polymorphisms in different individual plants to determine if any variant or polymorphism is associated with a trait, specifically the flower density trait of interest.

As used herein a “polymorphism” is a particular type of variance that includes both natural and/or induced multiple or single nucleotide changes, short insertions, or deletions in a target nucleic acid sequence at a particular locus as compared to a related nucleic acid sequence. These variations include, but are not limited to, single nucleotide polymorphisms (SNPs), indel/s, genomic rearrangements, and gene duplications.

As used herein, the term “LOD score” or “logarithm (base 10) of odds” refers to a statistical estimate used in linkage analysis, wherein the score compares the likelihood of obtaining the test data if the two loci are indeed linked, to the likelihood of observing the same data purely by chance. The LOD score is a statistical estimate of whether two genetic loci are physically near enough to each other (or “linked”) on a particular chromosome that they are likely to be inherited together. A LOD score of 3 or higher is generally understood to mean that two genes are located close to each other on the chromosome. In terms of significance, a LOD score of 3 means the odds are 1,000:1 that the two genes are linked and therefore inherited together.

As used herein, the term “quantile-quantile” or “Q-Q” refers to a graphical method for comparing two probability distributions by plotting their quantiles against each other. If the two distributions being compared are similar, the points in the Q-Q plot will approximately lie on the line y=x. If the distributions are linearly related, the points in the Q-Q plot will approximately lie on a line, but not necessarily on the line y=x. Q-Q plots can also be used as a graphical means of estimating parameters in a location-scale family of distributions.

As used herein, a “causal gene” is the specific gene having a genetic variant (the “causal variant”) which is responsible for the association signal at a locus and has a direct biological effect on the flower density trait. In the context of association studies, the genetic variants which are responsible for the association signal at a locus are referred to as the “causal variants”. Causal variants may comprise one or more “causal polymorphisms” that have a direct biological effect on the phenotype.

The term “nucleic acid” encompasses both ribonucleotides (RNA) and deoxyribonucleotides (DNA), including cDNA, genomic DNA, isolated DNA and synthetic DNA. The nucleic acid may be double-stranded or single-stranded. Where the nucleic acid is single-stranded, the nucleic acid may be the sense strand or the antisense strand. A “nucleic acid molecule” or “polynucleotide” refers to any chain of two or more covalently bonded nucleotides, including naturally occurring or non-naturally occurring nucleotides, or nucleotide analogs or derivatives. By “RNA” is meant a sequence of two or more covalently bonded, naturally occurring or modified ribonucleotides. The term “DNA” refers to a sequence of two or more covalently bonded, naturally occurring or modified deoxyribonucleotides. By “cDNA” is meant a complementary or copy DNA produced from an RNA template by the action of RNA-dependent DNA polymerase (reverse transcriptase).

In some embodiments, the nucleic acid molecules of the invention may be operably linked to other sequences. By “operably linked” is meant that the nucleic acid molecules, such as those comprising the QTL of the invention or genes identified herein, and regulatory sequences are connected in such a way as to permit expression of the proteins when the appropriate molecules are bound to the regulatory sequences. Such operably linked sequences may be contained in vectors or expression constructs which can be transformed or transfected into plant cells or plants for expression. A “regulatory sequence” refers to a nucleotide sequence located either upstream, downstream or within a coding sequence. Generally regulatory sequences influence the transcription, RNA processing or stability, or translation of an associated coding sequence. Regulatory sequences include but are not limited to: effector binding sites, enhancers, introns, polyadenylation recognition sequences, promoters, RNA processing sites, stem-loop structures, translation leader sequences and the like.

The term “promoter” refers to a DNA sequence that is capable of controlling the expression of a nucleic acid coding sequence or functional RNA. A promoter may be based entirely on a native gene, or it may be comprised of different elements from different promoters found in nature. Different promoters are capable of directing the expression of a gene at different stages of development, or in response to different environmental or physiological conditions. An “inducible promoter” is promoter that is active in response to a specific stimulus. Several such inducible promoters are known in the art, for example, chemical inducible promoters, developmental stage inducible promoters, tissue type specific inducible promoters, hormone inducible promoters, environment responsive inducible promoters.

The term “isolated”, as used herein means having been removed from its natural environment. Specifically, the nucleic acid or gene(s) identified herein may be isolated nucleic acids or gene(s), which have been removed from plant material where they naturally occur.

The term “purified”, relates to the isolation of a molecule or compound in a form that is substantially free of contamination or contaminants. Contaminants are normally associated with the molecule or compound in a natural environment, purified thus means having an increase in purity as a result of being separated from the other components of an original composition. The term “purified nucleic acid” describes a nucleic acid sequence that has been separated from other compounds including, but not limited to polypeptides, lipids, and carbohydrates which it is ordinarily associated with in its natural state.

The term “complementary” refers to two nucleic acid molecules, e.g., DNA or RNA, which are capable of forming Watson-Crick base pairs to produce a region of double-strandedness between the two nucleic acid molecules. It will be appreciated by those of skill in the art that each nucleotide in a nucleic acid molecule need not form a matched Watson-Crick base pair with a nucleotide in an opposing complementary strand to form a duplex. One nucleic acid molecule is thus “complementary” to a second nucleic acid molecule if it hybridizes, under conditions of high stringency, with the second nucleic acid molecule. A nucleic acid molecule according to the invention includes both complementary molecules.

As used herein a “substantially identical” or “substantially homologous” sequence is a nucleotide sequence that differs from a reference sequence only by one or more conservative substitutions, or by one or more non-conservative substitutions, deletions, or insertions located at positions of the sequence that do not destroy or substantially alter the activity of the polypeptide encoded by the nucleic acid molecule. Alignment for purposes of determining percent sequence identity can be achieved in various ways that are within the knowledge of those with skill in the art. These include using, for instance, computer software such as ALIGN, Megalign (DNASTAR), CLUSTALW or BLAST software. Those skilled in the art can readily determine appropriate parameters for measuring alignment, including any algorithms needed to achieve maximal alignment over the full length of the sequences being compared. In one embodiment of the invention there is provided for a polynucleotide sequence that has at least about 80% sequence identity, at least about 90% sequence identity, or even greater sequence identity, such as about 95%, about 96%, about 97%, about 98% or about 99% sequence identity to the sequences described herein.

Alternatively, or additionally, two nucleic acid sequences may be “substantially identical” or “substantially homologous” if they hybridize under high stringency conditions. The “stringency” of a hybridisation reaction is readily determinable by one of ordinary skill in the art, and generally is an empirical calculation which depends upon probe length, washing temperature, and salt concentration. In general, longer probes required higher temperatures for proper annealing, while shorter probes require lower temperatures. Hybridisation generally depends on the ability of denatured DNA to re-anneal when complementary strands are present in an environment below their melting temperature. A typical example of such “stringent” hybridisation conditions would be hybridisation carried out for 18 hours at 65° C. with gentle shaking, a first wash for 12 min at 65° C. in Wash Buffer A (0.5% SDS; 2×SSC), and a second wash for 10 min at 65° C. in Wash Buffer B (0.1% SDS; 0.5% SSC).

Nucleotide positions of polymorphisms described herein are provided with reference to the corresponding position on the Cannabis sativa (assembly cs10) representative genome, provided as RefSeq assembly accession: GCF_900626175.2 on NCBI, loaded on 14 Feb. 2019, referred to herein as “cs10 reference genome” or “cs10 genome”.

### Methods of Identifying a QTL or Haplotype Responsible for the Flower Density Phenotype and Molecular Markers Therefor

In some embodiments, methods are provided for identifying a QTL or haplotype responsible for flower density and for selecting plants that have the flower density trait of interest, thereby to identify the QTL or haplotype responsible for the trait. In some embodiments, the methods may comprise the steps of:
a. Identifying a plant that displays the increased/decreased flower density trait within a breeding program.
b. Establishing a population by crossing the identified plant to itself (selfing) or a recipient parent plant.
C. Genotyping the resultant F1, or subsequent populations, for example by sequencing methods.
d. Performing association studies, including phenotyping and linkage analysis, to discover QTLs and/or polymorphisms contained within the QTL.
e. Optionally, identifying Cannabis paralogs of previously characterized genes that may be involved in conferring the increased/decreased flower density phenotype.
f. Developing molecular markers that detect one or more polymorphisms linked to QTLs, alleles within these QTLs, or existing or induced polymorphisms.
g. Validating the molecular markers by determining the linkage disequilibrium between the marker and the flower density trait.

### Trait Development and Introgression

In some embodiments, methods are provided for marker assisted breeding (MAB) or marker assisted selection (MAS) of plants which have the flower density QTL or display the flower density trait of interest. The methods may comprise the steps of:
a. Identifying a plant that displays the flower density trait of interest or which contains a flower density QTL as defined herein.
b. Establishing a population by crossing the identified plant to itself (selfing) or another recipient parent plant.
C. Genotyping and phenotyping the resultant F1, or subsequent, populations, for example by sequencing methods.
d. Performing association studies, inputting phenotype and genotype information to identify genomic regions enriched with polymorphisms associated with the flower density trait of interest, to discover QTLs and/or polymorphisms contained within the QTL.
e. Optionally, identifying Cannabis paralogs of previously characterized genes that may be involved in the flower density trait.
f. Developing molecular markers that detect one or more polymorphisms linked to QTLs, alleles within these QTLs, or existing or induced polymorphisms.
g. Using the molecular markers when introgressing the QTLs or polymorphisms into new or existing Cannabis varieties to select plants containing the flower density haplotype or the flower density trait of interest.

### QTLs and Marker Assisted Breeding

In some embodiments, during the breeding process, selection of plants displaying the flower density trait of interest or haplotype conferring the trait may be based on molecular markers designed to detect polymorphisms linked to genomic regions that control the trait of interest. In some embodiments, QTLs containing such elements are identified using association studies. Knowledge of the mode-of-action is not required for the functional use of these genomic regions in a breeding program. Identification of regions controlling unidentified mechanisms may be useful in obtaining plants with the flower density trait of interest, based on identification of polymorphisms that are either linked to, or found within QTLs that are associated with the flower density trait of interest using association studies.

### Construction of Breeding Populations

Breeding populations are the offspring of sexual reproduction events between two or more parents. The parent plants (F0) are crossed to create an F1 population each containing a chromosomal complement of each parent. In a subsequent cross (F2), recombination has occurred and allows for mostly independent segregation of traits in the offspring and importantly the reconstitution of recessive phenotypes that existed in only one of the parental lines.

According to some embodiments, QTLs that lead to the flower density trait of interest are identified within synthetic populations of plants capable of revealing dominant, recessive, or complex traits. In one embodiment of the invention, a genetically diverse population of Cannabis varieties, that are used to produce the synthetic population are integrate them into a breeding program by unnatural processes. In some embodiments, these processes result in changes in the genomes of the plants. The changes may include, but are not limited to, mutations and rearrangements in the genomic sequences, duplication of the entire genome (polyploidy), or activation of movement of transposable elements which may inactivate, activate or attenuate the activity of genes or genomic elements. According to one embodiment of the invention, the methods employed to integrate the plants into a breeding program include some or all of the following:
a. Growing plants in rich media or soils under artificial lighting;
b. Cloning of plants, often through a multitude of sub-cloning cycles;
C. Introduction of plants into in vitro, sterile growth environments, and subsequent removal to standard growth conditions;
d. Exposure to mutagens such as EMS, colchicine, silver nitrate, ethidium bromide, dinitroanalines, high concentrations mono or poly-chromatic light sources;
e. Growing plants under highly stressful conditions which include restricted space, drought, pathogen, atypical temperatures, and nutrient stresses.

### Flower Density Trait Association Studies and QTL Identification

In some embodiments, the synthetic populations created are either the offspring of the sexual reproduction or clones of plants in the breeding program such that genetic material of individuals in the synthetic populations is derived from one, or two, or more plants from the breeding program.

In one embodiment, plants identified within the synthetic population as having a trait of interest, such as the increased or decreased flower density trait, may be used to create a structured population for the identification of the genetic locus responsible for the trait. The structured population may be created by crossing one (selfing) or more plants and recovering the seeds from those plants.

Plants in the structured population may be fully genotyped using genome sequencing to identify genetic markers for use in the association study (AS) database. Association mapping is a powerful technique used to detect quantitative trait loci (QTLs) specifically based on the statistical correlation between the phenotype and the genotype. In this case the trait is the flower density trait. In a population generated by crossing, the amount of linkage disequilibrium (LD) is reduced between genetic marker and the QTL as a function of genetic distance in Cannabis varieties with similar genome structures. Simple association mapping is performed by biparental crosses of two closely related lines where one line has a phenotype of interest and the other does not. In some embodiments, advanced population structures may be used, including nested association mapping (NAM) populations or multi-parent advanced generation inter-cross (MAGIC) populations, however it will be appreciated that other population structures can also be effectively used. Biparental, NAM, or MAGIC structured populations can be generated and offspring, at F1 or later generations, may be maintained by clonal propagation for a desired length of time. In some embodiments, QTLs may be identified using the high-density genetic marker database created by genotyping the founder lines and structured population lines. This marker database may be coupled with an extensive phenotypic trait characterization dataset, including, for example, the flower density phenotype of the plants. Using the association studies described herein, together with accurate phenotyping, this method is able to identify genomic regions, QTLs and even specific genes or polymorphisms responsible for the flower density trait of interest that are directly introduced into recipient lines. Polygenic phenotypes may also be identified using the methods described herein.

In one embodiment, the structured population is grown to the time of harvest. To characterize the phenotypes of the lines they are clonally reproduced so the phenotypic data can be collected in feasible replicates.

### Genomic Selection

In some embodiments, during the breeding process, selection of plants by genomic selection (GS) may be conducted. Genomic selection is a method in plant breeding where the genome wide genetic potential of an individual is determined to predict breeding values for those individuals. In some embodiments, the accuracy of genomic selection is affected by the data used in a GS model including size of the training population, relationships between individuals, marker density, use of pedigree information, and inclusion of known QTLs.

In some embodiments, a QTL or a SNP known to be associated with a trait that contributes to selection criteria can improve the accuracy of genomic selection models. In some embodiments, a genomic selection model that incorporates flower density traits can be improved by the inclusion of the flower density QTL in the GS model. In some embodiments, the SNPs described in Table 2 or 3 may be useful in a genomic selection model, for example where genotypes with unknown phenotypes are evaluated using an approach like a random forest algorithm for prediction of the flower density trait, and particularly in combination, to improve the predictive power of the model.

### Molecular Markers to Detect Polymorphisms

As used herein, the term “marker” or “genetic marker” refers to any sequence comprising a particular polymorphism or haplotype described herein that is capable of detection. For example, a marker may be a binding site for a primer or set of primers that is designed for use in a PCR-based method to amplify and thus detect a polymorphism or haplotype. Alternatively, the marker may introduce a restriction enzyme recognition site, or result in the removal of a restriction enzyme recognition site. Plants can be screened for a particular trait based on the detection of one or more markers confirming the presence of the polymorphism. Marker detection systems that may be used in accordance with the present invention include, but are not limited to polymerase chain reaction (PCR) followed by sequencing, Kompetitive allele specific PCR (KASP), restriction fragment length polymorphisms (RFLPs) analysis, amplified fragment length polymorphisms (AFLPs), cleaved amplified polymorphic sequences (CAPS), or any other markers known in the art.

In some embodiments “molecular markers” refers to any marker detection system and may be PCR primers, or targeted sequencing primers such as those described in the examples below, more specifically the primers defined in Table 4. For example, PCR primers may be designed that consist of a reverse primer and two forward primers that are homologous to the part of the genome that contains a polymorphism but differ in the 3′ nucleotide such that the one primer will preferentially bind to sequences containing the polymorphism and the other will bind to sequences lacking it. The three primers are used in single PCR reactions where each reaction contains DNA from a Cannabis plant as a template. Fluorophores linked to the forward primers provide, after thermocycling, a different relative fluorescent signal for homozygous and heterozygous alleles containing the polymorphism and for those lacking the polymorphism, respectively.

In some embodiments, allele-specific primers may each harbor a unique tail sequence that corresponds with a universal FRET (fluorescence resonant energy transfer) cassette. For example, the primer specific to the SNP may be labelled with a FAM and the other specific primer with a HEX dye. During the PCR thermal cycling performed with these primers, the allele-specific primer binds to the genomic DNA template and elongates, so attaching the tail sequence to the newly synthesized strand. The complement of the allele-specific tail sequence is then generated during subsequent rounds of PCR, enabling the FRET cassette to bind to the DNA. Alleles are discriminated through the competitive binding of the two allele-specific forward primers. At the end of the PCR reaction a fluorescent plate is read using standard tools which may include RT-PCR devices with the capacity to detect florescent signals and is evaluated with commercial software.

If the genotype at a given polymorphism site is homozygous, one of the two possible fluorescent signals will be generated. If the genotype is heterozygous, a mixed fluorescent signal will be generated. By way of example, genomic DNA extracted from Cannabis leaf tissue at seedling stage can be used as a template for PCR amplifications with reaction mixtures containing the three primers. Final fluorescent signals can be detected by a thermocycler and analyzed using standard software for this purpose, which discriminates between individuals that are heterozygotes or homozygotes for either allele.

In some embodiments, molecular markers to one, two or more of the SNPs in the haplotype can be used to identify the presence of the QTL and by association, the flower density trait of interest.

Further, the QTL may include a number of individual polymorphisms in linkage disequilibrium, which constitute a haplotype and which, with high frequency, can be inherited from a donor parent plant as a unit. Therefore, in some embodiments, molecular markers can be utilized which have been designed to identify numerous polymorphisms which are in linkage disequilibrium with other polymorphisms, any of which can be used to effectively predict the phenotype of the offspring for the flower density trait of interest.

According to some embodiments, any polymorphism in linkage disequilibrium with the flower density QTL can be used to determine the flower density haplotype in a breeding population of plants, as long as the polymorphism is unique to the flower density trait of interest in the donor parent plant when compared to the recipient parent plant.

In some embodiments the desired trait is the increased flower density trait, and the donor parent plant may be a plant that has been genetically modified or selected to include an increased flower density QTL defined by a polymorphism associated with the decreased flower density trait, for example any, some, or all of the polymorphisms defined in Table 2 or 3 associated with the trait.

Alternatively, the desired trait may be the decreased- or intermediate flower density trait, and the donor parent plant may be a plant that has been genetically modified or selected to include a decreased- or intermediate flower density QTL defined by a polymorphism conferring the decreased- or intermediate flower density trait, for example any, some, or all of the polymorphisms defined in Table 2 or 3 associated with the trait.

In some embodiments, donor parent plants, as described above, are used as one of two parents to create breeding populations (F1) through sexual reproduction. Methods for reproduction that are known in the art may be used. The donor parent plant provides the trait of interest to the breeding population. The trait is made to segregate through the population (F2) through at least one additional crossing event of the offspring of the initial cross. This additional crossing event can be either a selfing of one of the offspring or a cross between two individuals, provided that each plant used in the F1 cross contains at least one copy of a desired QTL allele or haplotype.

In some embodiments, the flower density allele or flower density haplotype in plants to be used in the F1 cross is determined using the described molecular markers. In some embodiments, the resulting F2 progeny is/are screened for any of the flower density polymorphisms associated with the flower density trait of interest described herein.

The plants at any generation can be produced by asexual means like cutting and cloning, or any method that yields a genetically identical offspring.

Production of Cannabis Spp. Plants Having the Increased Flower Density Trait

It is a particular aim of the present invention to provide for the production of Cannabis spp. plants that have the increased flower density trait. Accordingly, in some embodiments, a Cannabis spp. plant that has the decreased flower density trait may be converted into a plant having an increased flower density trait according to the methods of the present invention by providing a breeding population where the donor parent plant contains an increased flower density QTL associated with the increased flower density trait and recipient parent plant either displays the decreased flower density phenotype or contains the decreased flower density QTL.

In some embodiments the decreased flower density phenotype may be removed from a recipient parent plant by crossing it with a donor parent plant having the increased flower density QTL. In some embodiments the donor parent plant has an increased flower density phenotype and a contains a contiguous genomic sequence characterized by one or more of the polymorphisms of Table 2 associated with the increased flower density allele or flower density haplotype conferring or associated with the increased flower density trait.

In some embodiments, the donor parent plant is any Cannabis spp. variety that is cross fertile with the recipient parent plant.

In some embodiments, MAS or MAB may be used in a method of backcrossing plants carrying the increased flower density trait to a recipient parent plant. For example, an F1 plant from a breeding population can be crossed again to the recipient parent plant. In some embodiments, this method is repeated.

In some embodiments, the resulting plant population is then screened for the flower density trait using MAS with molecular markers to identify progeny plants that contain one or more polymorphism, such as any of those described Table 2, indicating the presence of an allele of a QTL associated with the increased flower density phenotype. In another embodiment, the population of Cannabis plants may be screened by any analytical methods known in the art to identify plants with desired characteristics, specifically increased flower density.

Production of Cannabis Spp. Plants Having the Decreased- or Intermediate Flower Density Trait

In some embodiments, a Cannabis spp. plant that has the increased flower density trait may be converted into a plant having a decreased flower density trait or intermediate flower density trait according to the methods of the present invention by providing a breeding population where the donor parent plant contains a decreased flower density QTL associated with the decreased flower density trait, or an intermediate flower density QTL associated with the intermediate flower density trait, and the recipient parent plant either displays the increased flower density trait or contains the increased flower density QTL.

In some embodiments the increased flower density phenotype may be removed from a recipient parent plant by crossing it with a donor parent plant having the decreased- or intermediate flower density QTL. In some embodiments the donor parent plant has a decreased- or intermediate flower density phenotype and a contains a contiguous genomic sequence characterized by one or more of the polymorphisms of Table 2 or 3 associated with the decreased- or intermediate flower density allele or haplotype associated therewith.

In some embodiments, the donor parent plant is any Cannabis spp. variety that is cross fertile with the recipient parent plant.

In some embodiments, MAS or MAB may be used in a method of backcrossing plants carrying the decreased- or intermediate flower density trait to a recipient parent plant. For example, an F1 plant from a breeding population can be crossed again to the recipient parent plant. In some embodiments, this method is repeated.

In some embodiments, the resulting plant population is then screened for the flower density trait using MAS with molecular markers to identify progeny plants that contain one or more polymorphism, such as any of those described Table 2 or 3, indicating the presence of an allele of a QTL associated with the decreased- or intermediate flower density phenotype. In another embodiment, the population of Cannabis plants may be screened by any analytical methods known in the art to identify plants with desired characteristics, specifically the decreased- or intermediate flower density trait.

### Methods to Genetically Engineer Plants to Achieve the Flower Density Trait of Interest Using Mutagenesis or Gene Editing Techniques

Identifying QTLs, and individual polymorphisms, that correlate with a trait when measured in an F1, F2, or similar, breeding population indicates the presence of one or more causal polymorphisms in close proximity the polymorphism detected by the molecular marker. In some embodiments, the polymorphisms associated with the increased-, decreased-, or intermediate flower density trait are introduced into a plant by other means so that a trait, can be introduced into plants that would not otherwise contain associated causal polymorphisms or removed from plants that would otherwise contain associated causal polymorphisms.

Examples of causal polymorphisms for the flower density trait of interest include an A/G SNP at position 104261554 on chromosome NC_044370.1 with reference to the cs10 reference genome (position 685 of SEQ ID NO:45) and/or a T/C SNP at position 104263067 on chromosome NC_044370.1 with reference to the cs10 reference genome (position 1271 of SEQ ID NO: 45).

Similarly, a causal gene may be introduced into a plant, or disrupted in a plant, in order to obtain a plant having the flower density trait of interest. A causal gene has been identified herein, having the NCBI gene identity number LOC115702276 and encoding a homolog of a transcriptional corepressor LEUNIG isoform X1-X4 protein, from arabidopsis. Further, the polymorphisms detailed in Table 2 or 3 are molecular markers that can be used to indicate the presence of the causal polymorphisms in the plant.

The entire QTL or parts thereof which confer the flower density trait of interest described herein, or the causal gene(s), polymorphisms, or nucleic acid molecules described herein, may be introduced into the genome of a Cannabis plant to obtain plants with a flower density trait of interest, through a process of genetic modification known in the art, for example, but not limited to, heterologous gene expression using an expression cassette including a sequence encoding the QTL or part thereof, the gene(s), or the nucleic acids comprising the causal polymorphisms. The expression cassette may contain all or part of the QTL(s) or gene(s), including causal polymorphisms, such as the causal polymorphism A/G at position 685 of SEQ ID NO:45 and/or T/C at position 1271 of SEQ ID NO:45. In particular, with reference to the causal polymorphism at position 685 of SEQ ID NO:45, a plant having a homozygous genotype of AA has an increased flower density trait, a plant with a heterozygous genotype of AG has an intermediate flower density trait, and a plant with the homozygous genotype GG has a decreased flower density trait. Similarly, with respect to the causal polymorphism at position 1271 of SEQ ID NO:45, a plant having a homozygous genotype of TT has an increased flower density trait, a plant with a heterozygous TC genotype has an intermediate flower density trait, and a plant with a homozygous CC genotype has a decreased flower density trait.

The trait described herein may be removed from, or introduced into, the genome of a Cannabis plant to obtain plants that exclude or include the causal polymorphisms and the potential to display a desired flower density trait of interest through processes of genetic modification known in the art, for example, but not limited to, CRISPR-Cas9 targeted gene editing, TILLING, non-targeted chemical mutagenesis using e.g., EMS.

The present invention further provides methods for producing a modified Cannabis spp. plant using genome editing or modification techniques. For example, genome editing can be achieved using sequence-specific nucleases (SSNs) the use of which results in chromosomal changes, such as nucleotide deletions, insertions or substitutions at specific genetic loci, particularly those associated with the flower density trait of interest, more particularly a polymorphism causal for the flower density trait selected from a A/G SNP at position 685 of SEQ ID NO: 45 and/or a T/C SNP at position 1271 of SEQ ID NO:45. Non limiting examples of SSNs include zinc finger nucleases (ZFNs), TAL effector nucleases (TALENs), meganucleases, and clustered regularly interspaced short palindromic repeats (CRISPR)/CRISPR-associated protein (Cas) system. In some embodiments, non-limiting examples of Cas proteins suitable for use in the methods of the present invention include Csnl, Cpfl, Cas9, Cas 12, Cas 13, Cas 14, CasX, and combinations thereof. In one embodiment, a modified Cannabis spp. plant having a flower density trait of interest is generated using CRISPR/Cas9 technology, which is based on the Cas9 DNA nuclease guided to a specific DNA target by a single guide RNA (sgRNA). For example, the genome modification may be introduced using guide RNA, e.g., single guide RNA (sgRNA) designed and targeted to introduce a polymorphism associated with the flower density trait of interest, such as a polymorphism causal for the flower density trait selected from a A/G SNP at position 685 of SEQ ID NO:45 and/or a T/C SNP at position 1271 of SEQ ID NO:45.

DNA introduction into the plant cells can be performed using Agrobacterium infiltration, virus-based plasmid delivery of the genome editing molecules and mechanical insertion of DNA (PEG mediated DNA transformation, biolistics, etc.). In some embodiments, the Cas9 protein may be directly inserted together with a gRNA (ribonucleoprotein-RNP's) in order to bypass the need for in vivo transcription and translation of the Cas9+gRNA plasmid in planta to achieve gene editing. In one embodiment, a genome edited plant may be developed and used as a rootstock, so that the Cas protein and gRNA can be transported via the vasculature system to the top of the plant and create the genome editing event in the scion.

According to one embodiment of the present invention, the method of genetically modifying a plant may be achieved by combining the Cas nuclease (e.g., Cas9, Cpf 1) with a predefined guide RNA molecule (gRNA). The gRNA is complementary to a specific DNA sequence targeted for editing in the plant genome and which guides the Cas nuclease to a specific nucleotide sequence. The predefined gene-specific gRNAs may be cloned into the same plasmid as the Cas gene and this plasmid is inserted into plant cells as described above.

In some embodiments, once the gRNA molecule and Cas9 nuclease reach the specific predetermined DNA sequence, the Cas9 nuclease cleaves both DNA strands to create double stranded breaks leaving blunt ends. This cleavage site is then repaired by the cellular non homologous end joining DNA repair mechanism resulting in insertions or deletions which introduce a mutation at the cleavage site.

In one embodiment, a deletion form of the mutation may consist of at least 1 base pair deletion. As a result of this base pair deletion, the gene coding sequence for the putative gene(s) responsible for the flower density trait of interest, such as the genes described in Table 7, or more particularly a gene having the NCBI gene identity number LOC115702276 and encoding a homolog of a transcriptional corepressor LEUNIG isoform X1-X4 protein (SEQ ID NO:45), is disrupted and the translation of the encoded protein is compromised by the disruption of a start codon, introduction of a premature stop codon or disruption of a functional or structural property of the protein.

In another embodiment, the flower density trait of interest in Cannabis spp. plants may be introduced by generating gRNA with homology to a specific site of predetermined genes in the Cannabis genome or a QTL defined herein. In one embodiment the gene may be one or more of the genes described in Table 7 herein, or more particularly a gene having the NCBI gene identity number LOC115702276 and encoding a homolog of a transcriptional corepressor LEUNIG isoform X1-X4 protein (SEQ ID NO:45). This gRNA may be sub-cloned into a plasmid containing the Cas9 gene, and the plasmid inserted into the Cannabis plant cells. In this way site specific mutations in the QTL are generated, including the SNPs associated with the flower density trait of interest described in Table 2 or 3, and in particular a causal polymorphism, more particularly a A/G SNP at position 685 of SEQ ID NO:45 and/or a T/C SNP at position 1271 of SEQ ID NO:45, thus effectively introducing the flower density trait of interest into the genome edited plant.

In some embodiments, a modified Cannabis spp. plant exhibiting an increased flower density trait may be obtained using the targeted genome modification methods described above, wherein the plant comprises a targeted genome modification to introduce one or more polymorphisms associated with the increased flower density trait defined in Table 2 or 3, wherein the modification effects the increased flower density trait. In a preferred embodiment, the plant comprises a targeted genome modification to introduce a G>A SNP at position 685 of SEQ ID NO:45 and/or a C>T SNP at position 1271 of SEQ ID NO:45, to obtain a modified Cannabis spp. plant exhibiting an increased flower density trait.

In some embodiments, for example where the flower density trait of interest is a decreased flower density trait, the genetic modification may be introduced using gene silencing, a process by which the expression of a specific gene product is lessened or attenuated. Gene silencing can take place by a variety of pathways, including by RNA interference (RNAi), an RNA dependent gene silencing process. In one embodiment, RNAi may be achieved by the introduction of small RNA molecules, including small interfering RNA (siRNA), microRNA (miRNA) or short hairpin RNA (shRNA), which act in concert with host proteins (e.g., the RNA induced silencing complex, RISC) to degrade messenger RNA (mRNA) in a sequence-dependent fashion. In particular, RNAi may be used to silence one or more of the putative causal genes described in Table 7 herein, or more particularly a gene having the NCBI gene identity number LOC115702276 and encoding a homolog of a transcriptional corepressor LEUNIG isoform X1-X4 protein (SEQ ID NO:45). Such RNAi molecules may be designed based on the sequence of these genes. These molecules can vary in length (generally 18-30 base pairs) and may contain varying degrees of complementarity to their target mRNA in the antisense strand. Some, but not all, RNAi molecules have unpaired overhanging bases on the 5′ or 3′ end of the sense strand and/or the antisense strand. As used herein, the term “RNAi molecule” includes duplexes of two separate strands, as well as single strands that can form hairpin structures comprising a duplex region. The RNAi molecules may be encoded by DNA contained in an expression cassette and incorporated into a vector. The vector may be introduced into a plant cell using Agrobacterium infiltration, virus-based plasmid delivery of the vector containing the expression cassette and/or mechanical insertion of the vector (PEG mediated DNA transformation, biolistics, etc.).

Plants may be screened with the molecular markers as described herein to identify transgenic individuals with the flower density trait of interest or having a flower density QTL or polymorphism(s), following the genetic modification.

In some embodiments, Cannabis spp. plants having one or more of the polymorphisms of Table 2 or 3 associated with the flower density QTL or linked thereto are provided. More particularly, Cannabis spp. plants having a causal polymorphism, more particularly a A/G SNP at position 685 of SEQ ID NO:45 and/or a T/C SNP at position 1271 of SEQ ID NO:45 are provided. The polymorphisms, including the causal polymorphism, may be introduced, for example, by genetic engineering. In some embodiments the one or more polymorphisms associated with the flower density trait of interest or linked thereto are introduced into the plants by breeding, such as by MAS or MAB, or genomic selection, as described herein.

The flower density QTL described herein, or genes identified herein responsible for effecting the flower density trait, may be under the control of, or operably linked to, a promoter, for example an inducible promoter. Such QTL or genes may be operably linked to the inducible promoter so as to induce or suppress the flower density trait or phenotype in the plant or plant cell.

Accordingly, in a further embodiment, Cannabis spp. plants comprising a flower density QTL described herein, including an increased flower density QTL, and intermediate flower density QTL or a decreased flower density QTL, or one or more polymorphisms associated therewith, are provided. In some cases, such plants are provided for with the proviso that the plant is not exclusively obtained by means of an essentially biological process.

The following examples are offered by way of illustration and not by way of limitation.

### Example 1

### Genome-Wide Association Studies (GWAS) of Flower Density in Mixed F2 Population in Cannabis

The inventors undertook a survey of flower density in a diverse population of Cannabis flowers, including hemp-type and resin-type plants. Plants were originally assembled and grown in a field trial in 2020 in Niederwil, Switzerland. The inventors noticed a large diversity of flower density in this diverse population. Many of the plants with distinct flower density were used for targeted crosses and their progeny were selfed to obtain a number of F2 populations. During outdoor field trials in 2021, 11 of these F2 populations were grown to maturity and characterized for flower density in harvested dried flower in order to better understand the genetic basis of this trait (Table 1).

TABLE 1
Pedigree table showing the 11 F2 populations used in the
GWA study with the population identification number,
the average density of the population, standard deviation
of the average density of the population (StDev) and
the number of plants comprising the population.

| | | | Number |
|---|---|---|---|
| PopulationID | Average Density | StDev | of plants |
| 21 002 001 | 0.0964940728757299 | 0.0148453767797456 | 97 |
| 21 002 003 | 0.0986994410921798 | 0.0231710764449001 | 62 |
| 21 002 004 | 0.112493596478252 | 0.0167442738292071 | 57 |
| 21 002 012 | 0.0973792011406162 | 0.0156304447615856 | 38 |
| 21 002 014 | 0.1183432374269 | 0.0206174321180781 | 20 |
| 21 002 028 | 0.130956237983207 | 0.0225780599161157 | 48 |
| 21 002 035 | 0.129573477561819 | 0.0229432307930849 | 62 |
| 21 002 036 | 0.11146675632578 | 0.0237302544212673 | 50 |
| 21 002 038 | 0.138922982370411 | 0.028871150324261 | 41 |
| 21 002 041 | 0.109009762635272 | 0.0172844224512924 | 31 |
| 21 002 046 | 0.0974231057439194 | 0.018674709893994 | 53 |

The inventors observed the emergence of inflorescence in an outdoor field trial of each of the 11 F2 populations. In order to identify genetic regions associated with flower density in Cannabis flowers, these 11 F2 populations were assessed for flower density. Plants were harvested at maturity between October and November 2021. The apical inflorescence was cut to an approximate size of 35 cm, trimmed of its leaves, and freeze-dried until it contained a residual humidity level of between 7-10%. The dried flowers were weighed, the presence of seeds or physical damage were noted.

Because measuring flower density in Cannabis has not been undertaken the inventors devised a novel approach to obtain a metric to evaluate flower density. After weighing, the flowers underwent image capture followed by analysis. Flowers were placed on a blue surface and a picture was taken with a Canon EOS M6 II using a Canon EF-M 11-22 mm f/4-5.6 IS STM lens. After image acquisition, image segmentation and analysis were performed using custom ImageJ macros on a FIJI system (v.2.35). Images were first duplicated, with the first image being color segmented using the Hue values as a threshold. The particles on this segmented 8-bit image were subsequently size and position selected (particles with at least 1000 pixels, excluding particles on the edge). The resulting regions of interest (Rols) where considered without holes. The largest Rol was subsequently transferred to the colored copy of the image and used as a mask. Measurements of all specified values are calculated using this mask. Measurements include area, perimeter, Feret's diameter, shape and color descriptors of the flower.

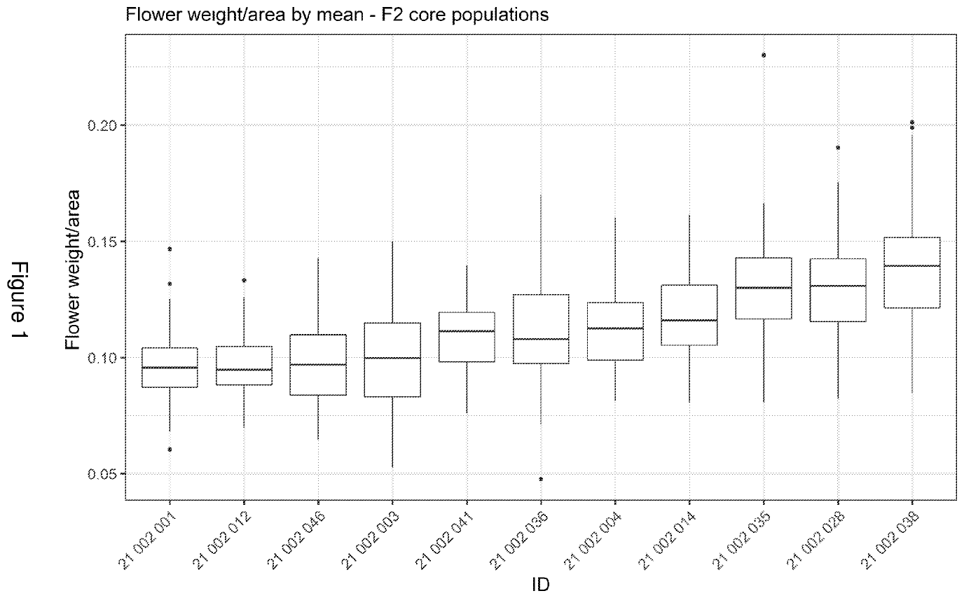

A filtering step was applied to remove outliers, including flowers noted to contain seed, and flowers with a length smaller than 30 cm. After filtering, flower density (g/cm²) was calculated by dividing flower weight by flower area. The flower density found for these F2 populations was found to vary considerably (FIG. 1). On average, population 21002001 was found to have the lowest flower density at 0.0965 g/cm², while population 21002038 was found to have the highest flower density at 0.1389 g/cm², representing an approximately 44% increase in flower density.

It is noted that the evaluation of the trait expression and segregation patterns is complicated by the influence of environmental factors on quantitative traits such as flower density. By conducting these experiments in a randomized field trial, the inventors sought to minimize positional effects in the field.

DNA was extracted from about 70 mg of leaf discs from all the plants evaluated in these 11 F2 populations, using an adapted kit with “sbeadex” magnetic beads by LGC Genomics, which was automated on a KingFisher Flex with 96 Deep-Well Head by Thermo Fisher Scientific.

The extracted DNA served as a template for the subsequent library preparation for sequencing. The library pools were prepared according to the manufacturer's instructions (AgriSeq™ HTS Library Kit-96 sample procedure from Thermo Fisher Scientific). Targeted sequencing of a custom SNP marker panel based on the Cannabis Sativa CS10 reference genome was carried out on the Ion Torrent system by Thermo Fisher Scientific. The primers for the SNPs identified in this study are all provided in Table 4. The library pool was loaded onto Ion 550 chips with Ion Chef and sequenced with Ion GeneStudio S5 Plus according to the manufacturer's instructions (Ion 550™ Kit from Thermo Fisher Scientific).

Targeted DNA sequenced from all 11 F2 populations segregating for the flower density trait, a population of 551 individuals, was used in a genome-wide association analysis (GWAS) to detect significant associations between genotypic information derived from targeted resequencing of the custom SNP marker panel designed based on the sequences provided in Table 3 and flower density (g/cm²).

The genotypic matrix was filtered for SNPs having more than 30% missing values within the population and a minor allele frequency lower than 5%. This resulted in 3858 SNP markers after filtering. The GWAS was performed using GAPIT version 3 (Wang and Zhang, 2021) with a Mixed Linear Model (MLM) to account for population structure. SNPs surpassing a Bonferroni-corrected LOD of 4.88 (−log₁₀ (0.05/number of markers)) were considered to have a significant association with trait variation.

SNPs showing a significant association with flower density, with a LOD value greater than 4.88, were found only on chromosome NC_044370.1 with reference to the Cannabis Sativa CS10 genome and are listed in Table 2. The homozygous allele of the SNPs in Table 2 that can distinguish a Cannabis plant that will produce a denser flower are listed (marked with an asterisk), along with their position. The alternative allele in this case indicates plants that will produce a less dense flower.

From the results of the GWA, the inventors identified one QTL based on the SNPs identified as being associated with flower density in the mixed F2 population listed in Table 2. The QTL is defined by the significantly associated SNPs on chromosome NC_044370.1, ranging from position 102037098 to 104628858. SNP “common_563” at position 103136879 on chromosome NC_044370.1 was found to have the strongest association with the flower density trait. The inventors have established SNP identifiers of a haplotype for increased flower density, decreased flower density, and an intermediate state, given in Table 2. The homozygous allele state marked with an asterisk indicates the allele associated with increased flower density. The homozygous unmarked allele indicates the allele associated with decreased flower density. While the heterozygous allele is associated with the intermediate flower density trait.

Because flower density is a quantitative trait, the inventors propose that the SNP based haplotypes identified will predispose the Cannabis plant for the flower density trait, however environmental and epistatic effects may influence the full expression of the trait. Nevertheless, for the purposes of identifying plants with the haplotype for the described flower density traits, the QTL and in particular the SNP markers identified are sufficient, based on the broad genetic diversity of the F2 populations used.

Surprisingly, when conducting GWA for flower density on the individual F2 populations alone, no significantly associated SNPs were detected. This indicates that the genetic diversity of the combined F2 populations, the phenotypic diversity of the combined F2 populations, use of population structure in the Mixed Linear Model, and the size of the combined populations contributed together to the identification of the flower density QTL.

TABLE 2
SNPs associated with flower density in Cannabis from a mixed F2 population on Chromosome
NC_044370.1. The presence of the increased flower density is predicted by the occurrence
of the indicative allele (marked with *). The position of the SNPs is provided with reference
to the CS10 reference genome as described herein. The three allele possibilities for the
SNP are listed as Allele 1, Allele 2, and Alle 3. The corresponding mean density for each
allele is given as Mean 1, Mean 2, and Mean 3. The number of plants that contribute to
each mean value is given as Count 1, Count 2, and Count 3. The LOD score based on the MLM
model for the association between each SNP and flower density is given as well.

| SNP Name | Position | LOD | Allele 1 | Allele 2 | Allele 3 | Mean 1 |
|---|---|---|---|---|---|---|
| common_563 | 103136879 | 6.88683344 | AT | TT | AA* | 0.1152096 |
| GBScompat_rare_14 | 103684608 | 6.26817964 | AC | CC* | AA | 0.11114352 |
| common_573 | 103875265 | 5.94392408 | AG | GG* | AA | 0.11107294 |
| GBScompat_common_102 | 103509580 | 5.59236425 | AA | AG | GG* | 0.10250386 |
| rare_66 | 102037098 | 5.09194002 | AA | AT | TT* | 0.09546206 |
| common_583 | 104628858 | 4.83014586 | AG | GG* | AA | 0.11608229 |

| SNP Name | Mean 2 | Mean 3 | Count 1 | Count 2 | Count 3 |
|---|---|---|---|---|---|
| common_563 | 0.09754458 | 0.12441587 | 230 | 166 | 85 |
| GBScompat_rare_14 | 0.11808181 | 0.09971385 | 273 | 118 | 108 |
| common_573 | 0.11799558 | 0.10672888 | 217 | 80 | 206 |
| GBScompat_common_102 | 0.11166917 | 0.11992436 | 146 | 241 | 94 |
| rare_66 | 0.1102269 | 0.11987296 | 42 | 308 | 94 |
| common_583 | 0.12264017 | 0.10270109 | 186 | 74 | 249 |

The SNPs identified are further characterized in Table 3 with reference to the alleles present, where the reference allele “Ref” is the allele found in the CS10 reference genome, while the alternative allele “Alt” indicates the alternative base found at this locus. The SNPs as well as context sequences in Table 3 are defined with reference to the CS10 reference genome.

Additional SNPs, “pos_1093_A”, “Leun229” and “Leun424” identified in Examples 2 and 3 below are also defined in Table 3. SNP “pos_1093_A” was initially proposed as a causal SNP on chromosome NC_044370.1 at position 103015433, a C/T polymorphism. In this case the reference allele (C) is also provided with reference to the CS10 genome, where the alternative is the allele state that would lead to the increased flower density phenotype (T). SNP “Leun229” is a proposed causal SNP on chromosome NC_044370.1 at position 104261554, a A/G polymorphism. In this case the reference allele (A) is also provided with reference to the CS10 genome, where the alternative is the allele state that would lead to the decreased flower density phenotype (G). SNP “Leun424” is a proposed causal SNP on chromosome NC_044370.1 at position 104263067, a T/C polymorphism. In this case the reference allele (T) is also provided with reference to the CS10 genome, where the alternative is the allele state that would lead to the decreased flower density phenotype (C).

TABLE 3
Detailed information of each of the SNPs associated with flower density in Cannabis as
provided in Table 2, as well as potential causal SNPs investigated in Examples 2 and 3 below
(“pos_1093_A”, “Leun229” and “Leun424”). The reference allele “Ref” is the allele found in the
CS10 reference genome, while the alternative allele “Alt” indicates the alternative base found at
this locus. The allele that is associated with increased flower density is marked with an asterisk,
where a plant with the homozygous state of the allele has a propensity for increased flower
density, a plant with the homozygous state of the unmarked allele has a propensity for decreased
flower density and a plant with the heterozygous state of the allele has a propensity for
intermediate flower density. The SNPs as well as context sequences are provided with reference
to the CS10 reference genome as described herein. All of the sequences and alleles are provided
with reference to the plus strand.

| SNP Name | Ref | Alt | Context Sequence |
|---|---|---|---|
| common_563 | A* | T | GGAGTGTAAATGCCTACACAAAGAAAATTTTATGAATTAGATAAATC |
| | | | TTTCTTCACAACACAAACAGTTGATACTAAGAAATCAGCTAAAAGGT |
| | | | GAAATGAAAACTTGAAGCAAGAGCTACAAATAATTACCGTAGGCTG |
| | | | AATAAGAAGACAAGACTATAATGTATGCTGAAGCTCTTTTGATTGAA |
| | | | TACCACAGAGGTGA[A/T]GTACTCTTTGAAGCTCTGTATGGAACATG |
| | | | ATACCTGTTTTTATAAGGTTTGAAAGGTTTACCCTCACCCTGCAATA |
| | | | AAATTTCCAAAATTTGGTCCATATATTAAGAGCAATTACAACCAAAT |
| | | | GGAATGGCACTACTGAGGGTAGAAATAACAAAGCATTTCAGGATAA |
| | | | AAGTAAAGCTCAAGACATATCACAATTTAAGA (SEQ ID NO: 1) |
| GBScompat_ | C* | A | GAGTCTGTGTCAAAGGAAACTGATGTAGGGACCATTTTGGAGGATA |
| rare_14 | | | TCAAATGTGCCATCCAGGAGACACATGATACTCTCCACCAGCACGC |
| | | | AGTAAGTTGTGTTTCTGAAGATGTTCATTGTTCTGATGCCAGATCTG |
| | | | ATAGGCAGGTCAATCCAGAAGATGCTGGCTTAACTGCTGATAAGGA |
| | | | AATTTCTCTTTCTCAG[C/A]CAGCCAGGGAAAGTGAGCAGATTAGCA |
| | | | AAGATTTAGCTGCTGCCATTTCTCAGATCCATGATTTTGTAACCTTC |
| | | | TTGGGCAAAGAAGCTATGGTAGTCCACGACACTTTCACCGAAAGCG |
| | | | ATGGATTCAGCAAGAGAATTGAAGATTTCTCTGTGACCTATAATAAA |
| | | | GTCATGCACAGCAACCTAAGTTTACTTGATTTTG (SEQ ID NO: 2) |
| common_573 | G* | A | TTGACAACGTCCCACCTTTCTAATTGACTGATTTTGGACCACTGGGT |
| | | | TAGTTCTTGGGGAACAAGATGGAACATCAAGCCAAGCTGGAATTTT |
| | | | AGCAGTTGGTAACCTGCAATAGTAAAAGCTAAATTAAAAGGGTCCA |
| | | | ACATAATATTACAAGAGTAAACAAGCAAAAGCACTTACGGTGTACAT |
| | | | GTATCATTAACTCTA[G/A]CACCTTGTGAAATGGTTGATGACGGTCC |
| | | | AGGCAATGATCCCATTCCTTCAAATTAAAGAAGGATTCATTTTAATTT |
| | | | CCAAATAATGATGATAAAATGTGTTTATCAGAAAGACCATATACACT |
| | | | TACCAGCTTTCTTGGAGCTCAAAATAGGTGTGTTCTGAAATGGGGT |
| | | | ACCATAAACTACGTTCAGATCATAGTCAAAAC (SEQ ID NO: 3) |
| GBScompat_ | G* | A | TTCACGGTTTCCACCCCCTCCGCCGGTTCCAGCTCCCTGTCCACC |
| common_102 | | | GTCACAGGTAAATGCTTCTGCGAAAGACATAAATTACTACAAGAGC |
| | | | TTGATTCAGCAACACGGAGGAGACAGGCAAGAGAATCTGAACCCT |
| | | | CCACAATATGGTAACCGTTATAATCACCAATCAGCGATGAGCCAGG |
| | | | AAATAGTTAACAGCCCTAA[G/A]CCGAGAGAGTCAAAGCCGAAAATA |
| | | | ATGAAACCTTGCATATATTTTAATAGCTCAAGGGGCTGCCGGCATG |
| | | | GAGCCAATTGTGCGTATCAGCATGATGTATCATACCAGCAGCGTGG |
| | | | TAGTTCCATGTCAGAGGTGCAGAATGCAAAGAGAACCAAATTTGAT |
| | | | AGAGAAATTAGCAGTTAAAAGTTGTAGTTCCCGTTTGTA (SEQ ID |
| | | | NO: 4) |
| rare_66 | A | T* | ATCAAGTAACTGCAAGTTGGGAATAGACCAGAGTGAACCAGGAAGA |
| | | | GCACCATCGAGTTTATTGTCACTCAGAACCACAACCCGCAGCTGAG |
| | | | AAAGGCTTGAAAACAAGCCCTTTGGCAATGGACCCTCTAGACCATT |
| | | | AGCACTAATATCAATAACCCTCAAGCTCCTCAAACCCGTGAATTCTT |
| | | | TAGGAAATGACCCAGT[A/T]AAAAAATTCTTGCTACAATTTAGCTCAA |
| | | | CAAGTCGAGAAAGTTTATTCAACTGAACAGGTATAGAAGCGGTAAG |
| | | | ACTATTATCAGAAGTACTTAAAAACTCAAGTCTCGAGAGGTTACCTA |
| | | | AACCTGATGGAATCGACCCAGAAAAGAAATTTGAGGAGAGGTCAAG |
| | | | AGTAGTAAGATTTCGAAGTGAGGTAAACTCGAAC (SEQ ID NO: 5) |
| common_583 | G* | A | CAGGGAAGCTCTGTGTACATATAAATTCAGCAATGAGGAAGCAGAT |
| | | | AAAATTCTTGGAAAAGCTTTTGGCCAGATACACTCGCCTTACTGGG |
| | | | GTGAAGAACGCAAAAAGGTTGTTCCGAAACTTGAACTTGTGAATGA |
| | | | AATTTTGGACTACTTAAGAAATTTGAACCTATCAGAAGATGATCTCG |
| | | | CGAAGGTGCTTAAAAA[G/A]TTTCCTGAAGTTCTTGGATGTGATCTA |
| | | | GAAGATGAGGTAAAAGTCAATGTGCAGGTTTTGGAGAAGGAATGG |
| | | | GAAATCAAAGGAAAAACTCTTCGAAACCTTCTTCTTCGAAATCCAAG |
| | | | ACTATTGGGTTACAACATTGACTGCAAGGGAGATTGTGCAGCACAA |
| | | | TGCACGCGATGCTGGGCTCGTTTTTAAGCTAAATCT (SEQ ID NO: 6) |
| pos_1093_A | C | T* | TTTGACTTGGGGACAAAAAGGTTGATCCAAGGGTGGGGTACGTCC |
| | | | CACAAT[C/T]CCTGGGACCTGAGTTTCAACTCGGATGTGTGGACTCG |
| | | | GTCCAAGAATTCCA (SEQ ID NO: 7) |
| Leun229 | A* | G | CTTGTTGGAAGTGATCCGCTCATGCGACAAAATCCAGGAACAGCTA |
| | | | ATGCCATGGCTACAAAGATGTATGAGGAGCGATTAAAGCTGCCTTC |
| | | | TCAAAGAGATCCTTTAGATGATGCAGCTATGAAGCAGCAAAGATTT |
| | | | GGTGAGAGTGTGGGCCAACTTTTGGATCCAACAAGTCAGACCTCCA |
| | | | TATTAAAGTCACCTGCA[A/G]CATCCAGCCAGCCATCAGGGCAAGTA |
| | | | TTGCATGGTTCAGCTGGAGCGATGTCTCCTCAAGTTCAAGCTCGAA |
| | | | ATCAACAATTGCCAGGGTCCACCCCGGACATAAAGCCTGAAACTAA |
| | | | TCCAGTTTTGAACCCCCGAGGTGCTGGTCAAGAAGGATCATTAGTA |
| | | | GGAATGCCCGGGTCAAATCAAGGAGGAAACAATTTGA (SEQ ID |
| | | | NO: 8) |
| Leun424 | T* | C | TTAGAATGCTTTTGAGTAACCGAAATTTGGGTCTTGTAAAGGATGGC |
| | | | TTTTCGAGTTCTGTTGGAGATGTGGTTCCCAATGTTGGATCCCAACT |
| | | | TCAAGGGAGTGGGGGTCATGTTTTGCCTCGTGGAGATACGGATAT |
| | | | GCTGATTAAGTTAAAAATGGCTCAACTACAGCAGCAGCAACAGCAA |
| | | | CAACAGAACAGTACAC[T/C]GTCACAGCAGCTACAGCAACATTCAAA |
| | | | TCAGCAGTCACAGAGTTCCAATCACAATCCACACCAAGATAAGATG |
| | | | GGTGGTGCTGGCAGTGTGACCATGGACGGTAGCATATCAAACTCC |
| | | | TTTCGAGGAAATGACCAGGGTTCAAAAAGCCAGACTGGTAGAAAGC |
| | | | GAAAACAGCCAGTGTCATCTTCGGGTCCTGCAAATAG (SEQ ID |
| | | | NO: 9) |

TABLE 4
Targeted sequencing primers (5′ to 3′) for the SNPs identified in Tables 2 and 3, as
described in the Examples.

| SNP Name | Primer1 Forward | Primer1 Reverse | Primer2 Forward | Primer2 Reverse |
|---|---|---|---|---|
| common_563 | TCTTTCTTCACAA | CCCTCAGTAGT | TCTTTCTTCACAA | ACCCTCAGTAGT |
| | CACAAACAGT | GCCATTCCA | CACAAACAGTT | GCCATTCC |
| | (SEQ ID NO: 10) | (SEQ ID NO: 11) | (SEQ ID NO: 12) | (SEQ ID NO: 13) |
| GBScompat_ | ATGTGCCATCCA | GCTGAATCCAT | GTGCCATCCAGG | GCTGAATCCATC |
| rare_14 | GGAGACAC | CGCTTTCGG | AGACACAT | GCTTTCGG |
| | (SEQ ID NO: 14) | (SEQ ID NO: 15) | (SEQ ID NO: 16) | (SEQ ID NO: 15) |
| common_573 | TGACAACGTCCC | GATCATTGCCT | GCCAAGCTGGAA | ACCCCATTTCAG |
| | ACCTTTCT | GGACCGTCA | TTTTAGCAGT | AACACACCT |
| | (SEQ ID NO: 17) | (SEQ ID NO: 18) | (SEQ ID NO: 19) | (SEQ ID NO: 20) |
| GBScompat_ | GTTCCAGCTCCC | GAACTACCACG | CTGTCCACCGTC | GAACTACCACGC |
| common_102 | TGTCCAC | CTGCTGGTA | ACAGGTAA | TGCTGGTA |
| | (SEQ ID NO: 21) | (SEQ ID NO: 22) | (SEQ ID NO: 23) | (SEQ ID NO: 22) |
| rare_66 | TCACTCAGAACC | CTGGGTCGATT | TCACTCAGAACC | TCTGGGTCGATT |
| | ACAACCCG | CCATCAGGT | ACAACCCG | CCATCAGG |
| | (SEQ ID NO: 24) | (SEQ ID NO: 25) | (SEQ ID NO: 24) | (SEQ ID NO: 26) |
| common_583 | GGCCAGATACAC | TGTGCTGCACA | GGCCAGATACAC | TGCACAATCTCC |
| | TCGCCTTA | ATCTCCCTT | TCGCCTTA | CTTGCAGT |
| | (SEQ ID NO: 27) | (SEQ ID NO: 28) | (SEQ ID NO: 27) | (SEQ ID NO: 29) |
| Leun229 | GCCAACTTTTGG | CATCCAGCCAG | | |
| | ATCCAACAAGTC | CCATCAGGGTA | | |
| | AGACCTCCATATT | TTTTTTTGTTTTT | | |
| | AAAGTCACCTGC | GTTTTTTTACTT | | |
| | A (SEQ ID NO: 30) | TTT (SEQ ID | | |
| | | NO: 31) | | |
| Leun424 | AAAAATGGCTCA | GTCACAGCAGC | | - |
| | ACTACAGCAGCA | TACAGCAACATT | | |
| | GCAACAGCAACA | CAAATCAGCAG | | |
| | ACAGAACAGTAC | TCACAGAGTTC | | |
| | AC (SEQ ID NO: 32) | CAATC (SEQ ID | | |
| | | NO: 33) | | |

In order to validate the effectiveness of the SNP “common_563”, for use in marker assisted selection, a collection of 67 diverse Cannabis varieties were grown to harvest in the field in a field trial in Niederwil, Switzerland in 2021 and 2022. The inventors chose high resin-type THC, high resin-type CBD and hemp-type varieties for the trial. Flowers were harvested and air dried, scoring was conducted by visual inspection and plants were determined to be high, medium or low density. The 67 Cannabis varieties tested comprised part of a sequence proprietary pangenome study. The inventors extracted the genotype information for the SNP “common_563” and compared this to the phenotypic information derived from the trial. The inventors found that SNP “common_563” had a 77.6% accuracy in selecting the correct phenotype based on genotype information, demonstrating its effectiveness in improving selection for flower density.

### Example 2

### Gene Identification Based on Alignment and Protein Function

There are presently no known genes identified in Cannabis that have been shown to regulate flower density or flower size in Cannabis. The genetic regulation of flower structure has been described and characterized in several plant species, however the multitude of different genes involved in this process does not easily allow the identification of flower density genes in Cannabis. The inventors considered genes that may influence apical and lateral meristem maintenance or genes that may play a role in regulating flower organ development in Cannabis. They next sought to identify putative genes that could encode proteins that may be responsible for increased or decreased flower density. Using the findings of the association studies they identified candidate genes at the QTL identified.

The inventors determined that SNP differences between Cannabis genomes could inform which genes play a role in the trait of interest. Short reads from sequenced lines were dereplicated with NGSReadsTreatment (version 1.3, Gaia et al. (2019)) and pre-processed with fastp (version 0.23.2, S. Chen et al. (2018)). Reads were aligned to the CS10 reference genome with Bowtie2 (version 2.3.5.1, with options -rg and -rg-id to add read-group identifiers, Langmead and Salzberg (2012)). Only unique alignments with a mapping quality of at least 10 were kept. SNPs were called with freebayes and filtered for a minimal quality of 20 (version v1.3.2-40-gcce27fc, parameters -p 2-min-coverage 20-g 30000-min-alternate-count 4-min-alternate-fraction 0.1 -min-mapping-quality 10-max-complex-gap-1, Garrison and Marth (2012)). SNPs were finally filtered for a coverage between 5 and 10,000 within each line and annotated with snpEff (version 4_3t, Cingolani et al. (2012)).

For each line, the inventors constructed a pseudogenome by incorporating its variants into the CS10 reference genome with vcf-consensus (Danecek et al. (2011)). CS10 annotation was lifted over, to align genes from a reference genome to a target genome, with liftoff (version 1.6.3, Shumate and Salzberg (2021)). Protein and cDNA sequences were extracted with custom scripts. Proteins and cDNA sequences for a given protein/transcript from all lines were aligned with muscle (v3.8.31, Edgar (2004)).

Proteins on sequence NC_044370.1 being located between 102100000 and 105660000 bp were extracted. Multiple alignments from protein sequences were converted to tables including the variant positions and protein variants were tested for correlation with the significant SNPs from the GWAS marker panel. Only proteins with significant associations were kept. These proteins were then used to extract all SNPs within the associated genes. SNPs were also tested for association with the significant SNPs from the GWAS marker panel. Only significant SNPs were kept. SNPs were further filtered for being polymorphic in at least half of the grandparent pairs used to generate the test populations and for having an effect on the amino acid sequence of the protein. The remaining 274 SNPs (Table 5) and 99 associated proteins with homologs in Arabidopsis (Table 6) were finally used as candidates.

TABLE 5
274 SNPs showing association with the flower density trait based on the alignments
detailed above. The chromosome and positions are provided with reference to the CS10
reference genome.

| Chromosome | Position | SNP |
|---|---|---|
| NC_44370.1 | 102139456 | C/T |
| NC_44370.1 | 102139810 | T/C |
| NC_44370.1 | 102184037 | A/T |
| NC_44370.1 | 102243326 | G/C |
| NC_44370.1 | 102243383 | T/C |
| NC_44370.1 | 102243753 | G/A |
| NC_44370.1 | 102243766 | C/A |
| NC_44370.1 | 102244011 | G/A |
| NC_44370.1 | 102244821 | A/G |
| NC_44370.1 | 102244956 | G/A |
| NC_44370.1 | 102244983 | G/C |
| NC_44370.1 | 102245328 | C/A |
| NC_44370.1 | 102245575 | A/C |
| NC_44370.1 | 102245598 | G/A |
| NC_44370.1 | 102245649 | A/C |
| NC_44370.1 | 102245677 | A/G |
| NC_44370.1 | 102296354 | AAG/AATTAG |
| NC_44370.1 | 102296895 | C/A |
| NC_44370.1 | 102303830 | C/T |
| NC_44370.1 | 102757575 | G/T |
| NC_44370.1 | 102757768 | G/A |
| NC_44370.1 | 102758017 | A/T |
| NC_44370.1 | 102758723 | C/G |
| NC_44370.1 | 102810741 | C/G |
| NC_44370.1 | 102811767 | G/C |
| NC_44370.1 | 102811804 | TATGATGATGATGATGA (SEQ ID NO: 34)/ |
| | | TATGATGATGATGATGATGA (SEQ ID NO: 35) |
| NC_44370.1 | 102948127 | G/A |
| NC_44370.1 | 102994894 | C/G |
| NC_44370.1 | 103014240 | T/C |
| NC_44370.1 | 103015433 | C/T |
| NC_44370.1 | 103015538 | G/A |
| NC_44370.1 | 103053354 | G/C |
| NC_44370.1 | 103054518 | TGTGG/TGTGGTGG |
| NC_44370.1 | 103055916 | T/C |
| NC_44370.1 | 103056214 | A/G |
| NC_44370.1 | 103083004 | C/T |
| NC_44370.1 | 103083030 | A/G |
| NC_44370.1 | 103122624 | T/A |
| NC_44370.1 | 103123085 | C/T |
| NC_44370.1 | 103123244 | C/T |
| NC_44370.1 | 103138060 | C/T |
| NC_44370.1 | 103142943 | G/T |
| NC_44370.1 | 103142945 | A/G |
| NC_44370.1 | 103185842 | A/C |
| NC_44370.1 | 103185887 | G/A |
| NC_44370.1 | 103185963 | C/A |
| NC_44370.1 | 103186132 | C/A |
| NC_44370.1 | 103212369 | CCTGCTGC/CCTGCTGCTGC (SEQ ID NO: 36) |
| NC_44370.1 | 103250315 | G/T |
| NC_44370.1 | 103250409 | CCT/CACCAGCT |
| NC_44370.1 | 103251261 | C/A |
| NC_44370.1 | 103251282 | A/G |
| NC_44370.1 | 103251378 | G/C |
| NC_44370.1 | 103251431 | C/T |
| NC_44370.1 | 103279889 | G/C |
| NC_44370.1 | 103280208 | G/A |
| NC_44370.1 | 103361249 | T/A |
| NC_44370.1 | 103361511 | G/A |
| NC_44370.1 | 103361833 | A/C |
| NC_44370.1 | 103381112 | G/T |
| NC_44370.1 | 103405168 | A/G |
| NC_44370.1 | 103417484 | TGT/TCGATACGT |
| NC_44370.1 | 103417645 | GGTCGTCGTCG (SEQ ID NO: 37) / GGTCGTCGTCGTCG (SEQ |
| | | ID NO: 38) |
| NC_44370.1 | 103418024 | C/G |
| NC_44370.1 | 103418140 | G/A |
| NC_44370.1 | 103444589 | A/G |
| NC_44370.1 | 103452974 | C/T |
| NC_44370.1 | 103469992 | G/A |
| NC_44370.1 | 103472275 | A/G |
| NC_44370.1 | 103472466 | A/T |
| NC_44370.1 | 103493158 | C/T |
| NC_44370.1 | 103494465 | G/C |
| NC_44370.1 | 103494942 | G/T |
| NC_44370.1 | 103494983 | A/G |
| NC_44370.1 | 103495144 | A/T |
| NC_44370.1 | 103495243 | A/G |
| NC_44370.1 | 103495318 | G/T |
| NC_44370.1 | 103508439 | T/A |
| NC_44370.1 | 103508933 | G/A |
| NC_44370.1 | 103509344 | CAT/CCAGCAGAT |
| NC_44370.1 | 103509529 | A/G |
| NC_44370.1 | 103515741 | A/C |
| NC_44370.1 | 103525437 | T/C |
| NC_44370.1 | 103525689 | G/C |
| NC_44370.1 | 103525713 | C/G |
| NC_44370.1 | 103536957 | T/C |
| NC_44370.1 | 103539936 | C/T |
| NC_44370.1 | 103578871 | A/G |
| NC_44370.1 | 103578880 | C/T |
| NC_44370.1 | 103578954 | G/A |
| NC_44370.1 | 103578969 | C/T |
| NC_44370.1 | 103578993 | G/A |
| NC_44370.1 | 103579024 | C/A |
| NC_44370.1 | 103579219 | A/T |
| NC_44370.1 | 103579228 | C/T |
| NC_44370.1 | 103579243 | G/A |
| NC_44370.1 | 103579267 | G/A |
| NC_44370.1 | 103579326 | T/C |
| NC_44370.1 | 103579335 | C/T |
| NC_44370.1 | 103579351 | C/G |
| NC_44370.1 | 103579356 | CGA/CA |
| NC_44370.1 | 103579376 | G/C |
| NC_44370.1 | 103579381 | C/T |
| NC_44370.1 | 103579390 | G/T |
| NC_44370.1 | 103579449 | T/G |
| NC_44370.1 | 103579473 | G/A |
| NC_44370.1 | 103579492 | C/T |
| NC_44370.1 | 103579503 | A/G |
| NC_44370.1 | 103579506 | G/A |
| NC_44370.1 | 103579528 | T/C |
| NC_44370.1 | 103579629 | C/T |
| NC_44370.1 | 103579666 | T/C |
| NC_44370.1 | 103579743 | A/T |
| NC_44370.1 | 103579791 | C/G |
| NC_44370.1 | 103579792 | T/C |
| NC_44370.1 | 103579836 | C/T |
| NC_44370.1 | 103579858 | C/G |
| NC_44370.1 | 103579889 | G/T |
| NC_44370.1 | 103579912 | G/T |
| NC_44370.1 | 103579936 | C/T |
| NC_44370.1 | 103579938 | T/C |
| NC_44370.1 | 103579963 | A/C |
| NC_44370.1 | 103580094 | G/A |
| NC_44370.1 | 103580128 | G/T |
| NC_44370.1 | 103580181 | G/A |
| NC_44370.1 | 103580188 | G/G |
| NC_44370.1 | 103580227 | C/T |
| NC_44370.1 | 103580265 | A/G |
| NC_44370.1 | 103607721 | G/T |
| NC_44370.1 | 103607848 | T/C |
| NC_44370.1 | 103607868 | C/G |
| NC_44370.1 | 103784014 | T/C |
| NC_44370.1 | 103795126 | C/A |
| NC_44370.1 | 103795185 | G/A |
| NC_44370.1 | 103864149 | G/A |
| NC_44370.1 | 103864157 | A/T |
| NC_44370.1 | 103864158 | C/G |
| NC_44370.1 | 103874457 | G/G |
| NC_44370.1 | 103874467 | T/C |
| NC_44370.1 | 103874873 | T/A |
| NC_44370.1 | 103875252 | T/C |
| NC_44370.1 | 103875420 | T/C |
| NC_44370.1 | 103896761 | T/C |
| NC_44370.1 | 103909148 | C/T |
| NC_44370.1 | 103909188 | G/C |
| NC_44370.1 | 103910178 | G/A |
| NC_44370.1 | 103910310 | G/G |
| NC_44370.1 | 103948540 | G/A |
| NC_44370.1 | 103972028 | A/G |
| NC_44370.1 | 103972040 | A/G |
| NC_44370.1 | 103972417 | G/A |
| NC_44370.1 | 103972420 | G/A |
| NC_44370.1 | 103972487 | T/G |
| NC_44370.1 | 103972496 | A/G |
| NC_44370.1 | 103972501 | G/A |
| NC_44370.1 | 103972687 | G/A |
| NC_44370.1 | 103972792 | C/T |
| NC_44370.1 | 103976335 | G/A |
| NC_44370.1 | 103976652 | G/C |
| NC_44370.1 | 103976868 | G/G |
| NC_44370.1 | 103976953 | T/C |
| NC_44370.1 | 103977141 | T/G |
| NC_44370.1 | 104013143 | A/T |
| NC_44370.1 | 104022680 | T/A |
| NC_44370.1 | 104023916 | A/T |
| NC_44370.1 | 104023933 | T/A |
| NC_44370.1 | 104025531 | A/A |
| NC_44370.1 | 104025619 | G/A |
| NC_44370.1 | 104025626 | T/C |
| NC_44370.1 | 104025656 | C/A |
| NC_44370.1 | 104046815 | C/A |
| NC_44370.1 | 104056367 | G/C |
| NC_44370.1 | 104056376 | T/C |
| NC_44370.1 | 104056582 | G/C |
| NC_44370.1 | 104056636 | A/G |
| NC_44370.1 | 104056668 | C/G |
| NC_44370.1 | 104056715 | C/G |
| NC_44370.1 | 104056727 | G/A |
| NC_44370.1 | 104056772 | G/A |
| NC_44370.1 | 104056862 | C/G |
| NC_44370.1 | 104056877 | T/C |
| NC 44370.1 | 104064757 | ACA/AAGATCA |
| NC_44370.1 | 104064764 | A/G |
| NC_44370.1 | 104095478 | A/G |
| NC_44370.1 | 104112723 | A/C |
| NC_44370.1 | 104112872 | A/T |
| NC_44370.1 | 104113787 | T/A |
| NC_44370.1 | 104114410 | A/T |
| NC_44370.1 | 104116030 | G/T |
| NC_44370.1 | 104116529 | A/G |
| NC_44370.1 | 104116614 | C/G |
| NC_44370.1 | 104116949 | T/G |
| NC 44370.1 | 104117019 | A/T |
| NC_44370.1 | 104117240 | G/C |
| NC_44370.1 | 104134539 | T/C |
| NC_44370.1 | 104134541 | G/A |
| NC_44370.1 | 104134548 | TGGTGACAT/CAGTGACAT |
| NC_44370.1 | 104134572 | C/T |
| NC_44370.1 | 104134602 | A/T |
| NC_44370.1 | 104134720 | G/C |
| NC_44370.1 | 104135117 | A/T |
| NC_44370.1 | 104135516 | T/C |
| NC_44370.1 | 104137802 | T/C |
| NC 44370.1 | 104181201 | T/C |
| NC_44370.1 | 104182166 | G/A |
| NC 44370.1 | 104188392 | T/A |
| NC_44370.1 | 104188477 | G/A |
| NC_44370.1 | 104227357 | C/C |
| NC_44370.1 | 104227818 | T/G |
| NC_44370.1 | 104261554 | A/G |
| NC_44370.1 | 104263067 | T/C |
| NC_44370.1 | 104288254 | T/C |
| NC_44370.1 | 104299729 | C/G |
| NC_44370.1 | 104300284 | TACCAACACCAACAT (SEQ ID NO: 39)/ |
| | | TACCAACACCAACACCAACAT (SEQ ID NO: 40) |
| NC_44370.1 | 104300307 | G/C |
| NC_44370.1 | 104300311 | C/T |
| NC_44370.1 | 104300320 | C/T |
| NC_44370.1 | 104300322 | C/T |
| NC_44370.1 | 104330417 | T/C |
| NC_44370.1 | 104335474 | C/G |
| NC_44370.1 | 104336919 | A/G |
| NC_44370.1 | 104336937 | C/G |
| NC_44370.1 | 104342834 | TTAATAATG/TTGATAATG |
| NC_44370.1 | 104343031 | CAT/CCT |
| NC 44370.1 | 104343358 | G/A |
| NC_44370.1 | 104395958 | A/T |
| NC_44370.1 | 104396027 | G/A |
| NC 44370.1 | 104398874 | A/C |
| NC 44370.1 | 104402081 | A/C |
| NC_44370.1 | 104402092 | C/T |
| NC_44370.1 | 104402659 | GAAAAAACG/GAAAAAACC |
| NC_44370.1 | 104404673 | G/C |
| NC_44370.1 | 104411138 | G/A |
| NC_44370.1 | 104431992 | C/T |
| NC_44370.1 | 104433383 | C/G |
| NC_44370.1 | 104439029 | C/A |
| NC_44370.1 | 104445602 | A/G |
| NC_44370.1 | 104445912 | C/T |
| NC_44370.1 | 104445914 | T/C |
| NC_44370.1 | 104445965 | A/G |
| NC 44370.1 | 104446314 | A/T |
| NC_44370.1 | 104446316 | G/A |
| NC_44370.1 | 104469216 | G/T |
| NC_44370.1 | 104491989 | C/G |
| NC_44370.1 | 104494754 | C/A |
| NC_44370.1 | 104494787 | T/A |
| NC_44370.1 | 104499738 | G/T |
| NC_44370.1 | 104500248 | G/T |
| NC_44370.1 | 104500457 | A/G |
| NC_44370.1 | 104500980 | G/T |
| NC 44370.1 | 104501436 | C/A |
| NC 44370.1 | 104529563 | C/T |
| NC 44370.1 | 104537125 | T/A |
| NC_44370.1 | 104537181 | A/G |
| NC_44370.1 | 104548164 | A/T |
| NC_44370.1 | 104569774 | G/C |
| NC_44370.1 | 104574867 | G/A |
| NC 44370.1 | 104575058 | T/C |
| NC_44370.1 | 104592770 | G/A |
| NC_44370.1 | 104593141 | G/C |
| NC_44370.1 | 104602628 | G/A |
| NC_44370.1 | 104603142 | C/T |
| NC 44370.1 | 104622680 | C/C |
| NC 44370.1 | 104684212 | C/T |
| NC 44370.1 | 104833572 | T/A |
| NC_44370.1 | 104844297 | T/C |
| NC_44370.1 | 104853559 | A/T |
| NC_44370.1 | 104853620 | A/C |
| NC_44370.1 | 104855772 | G/T |
| NC_44370.1 | 104855948 | C/T |
| NC_44370.1 | 104856960 | G/T |
| NC_44370.1 | 104857870 | C/T |
| NC_44370.1 | 104906962 | C/T |
| NC_44370.1 | 104930383 | ATTGTTGTTGTAGT (SEQ ID NO: 41)/ |
| | | ATTGTTGTTGTAGTTGTTGTTGTAGT (SEQ ID NO: 42) |
| NC_44370.1 | 104930430 | G/T |

TABLE 6
99 associated candidate proteins with homologs in Arabidopsis based on the alignments detailed above.

| | Arabidopsis | | |
|---|---|---|---|
| cs10 Protein ID | homolog | Name | Description |
| XP_030485190.1 | AT1G75620 | | F10A5.18 |
| XP_030485080.1 | AT1G75520 | SRS5 | Protein SHI RELATED SEQUENCE 5 |
| XP_030485438.1 | AT1G22040 | | F-box/kelch-repeat protein At1g22040 |
| XP_030485136.1 | AT1G75540 | BBX21 | B-box zinc finger protein 21 |
| XP_030485272.1 | AT4G38900 | | Basic-leucine zipper (BZIP) transcription factor |
| | | | family protein |
| XP_030485693.1 | AT4G32160 | EREL1 | PX domain-containing protein EREL1 |
| XP_030491076.1 | AT1G20180 | | UPF0496 protein |
| XP_030485009.1 | AT2G27190 | PAP12 | Fe(3+)—Zn(2+) purple acid phosphatase 12 |
| XP_030485684.1 | AT1G22680 | | T22J18.15 |
| XP_030486587.1 | AT1G75450 | CKX5 | Cytokinin dehydrogenase 5 |
| XP_030485274.1 | AT1G19880 | | Regulator of chromosome condensation (RCC1) |
| | | | family protein |
| XP_030485459.1 | AT1G22060 | | F-box and Leucine Rich Repeat domains |
| | | | containing protein |
| XP_030485630.1 | AT3G05270 | FPP3 | Filament-like plant protein 3 |
| XP_030485660.1 | AT1G22540 | NPF5.10 | Protein NRT1/PTR FAMILY 5.10 |
| XP_030485062.1 | AT1G75520 | SRS5 | Protein SHI RELATED SEQUENCE 5 |
| XP_030485614.1 | AT5G25610 | RD22 | BURP domain protein RD22 |
| XP_030485427.1 | AT4G08570 | HIPP24 | Heavy metal-associated isoprenylated plant protein 24 |
| XP_030485246.1 | AT5G42520 | BPC6 | BPC6 |
| XP_030484494.1 | AT1G19530 | | DNA polymerase epsilon catalytic subunit A |
| XP_030484973.1 | AT2G16430 | PAP10 | Purple acid phosphatase |
| XP_030485655.1 | AT1G35460 | BHLH80 | Transcription factor bHLH80 |
| XP_030485276.1 | AT1G19870 | IQD32 | Protein IQ-DOMAIN 32 |
| XP_030486615.1 | AT1G22110 | | F2E2.18 |
| XP_030485308.1 | AT1G75730 | | At1g75730-like |
| XP_030485623.1 | AT4G08320 | TPR8 | Tetratricopeptide repeat (TPR)-like superfamily protein |
| XP_030479550.1 | AT2G35000 | ATL9 | E3 ubiquitin-protein ligase ATL9 |
| XP_030485622.1 | AT1G77620 | | p-loop containing nucleoside triphosphate |
| | | | hydrolases superfamily protein |
| XP_030485450.1 | AT1G22060 | | F-box and Leucine Rich Repeat domains |
| | | | containing protein |
| XP_030479568.1 | AT4G23160 | CRK8 | cysteine-rich RLK (RECEPTOR-like protein kinase) 8 |
| XP_030485671.1 | AT5G44250 | | At5g44250 |
| XP_030485042.1 | AT1G75520 | SRS5 | Protein SHI RELATED SEQUENCE 5 |
| XP_030485055.1 | AT1G75520 | SRS5 | Protein SHI RELATED SEQUENCE 5 |
| XP_030485392.1 | AT1G22140 | | At1g22140/F2E2_13 |
| XP_030485070.1 | AT1G75520 | SRS5 | Protein SHI RELATED SEQUENCE 5 |
| XP_030485335.1 | AT4G08685 | SAH7 | SAH7 |
| XP_030485642.1 | AT1G35340 | | ATP-dependent protease La (LON) domain protein |
| XP_030485412.1 | AT4G08570 | HIPP24 | Heavy metal-associated isoprenylated plant protein 24 |
| XP_030485499.1 | AT1G77920 | TGA7 | Transcription factor TGA7 |
| XP_030485273.1 | AT4G38900 | | Basic-leucine zipper (BZIP) transcription factor family protein |
| XP_030485665.1 | AT1G34550 | EMB2756 | Protein of unknown function (DUF616) |
| XP_030485252.1 | AT5G42520 | BPC6 | BPC6 |
| XP_030485616.1 | AT5G24810 | | ABC1 family protein |
| XP_030485617.1 | AT5G24810 | | ABC1 family protein |
| XP_030479678.1 | AT1G05990 | CML7 | Calmodulin-like protein 7 |
| XP_030485032.1 | AT4G16190 | RD19C | Probable cysteine protease RD19C |
| XP_030484919.1 | AT1G09850 | XBCP3 | At1g09850 |
| XP_030485629.1 | AT3G05270 | FPP3 | Filament-like plant protein 3 |
| XP_030485585.1 | AT4G32551 | LUG | Transcriptional corepressor LEUNIG |
| XP_030479628.1 | AT1G22110 | | F2E2.18 |
| XP_030486614.1 | AT1G22110 | | F2E2.18 |
| XP_030485418.1 | AT4G08570 | HIPP24 | Heavy metal-associated isoprenylated plant protein 24 |
| XP_030485095.1 | AT1G75520 | SRS5 | Protein SHI RELATED SEQUENCE 5 |
| XP_030485279.1 | AT3G27230 | | At3g27230 |
| XP_030485479.1 | AT4G33680 | DAP | LL-diaminopimelate aminotransferase, chloroplastic |
| XP_030485298.1 | AT1G47840 | HXK3 | Hexokinase-like 1 protein |
| XP_030485625.1 | AT1G21830 | | At1g21820 |
| XP_030479564.1 | AT3G01410 | | Polynucleotidyl transferase, ribonuclease H-like |
| | | | superfamily protein |
| XP_030485505.1 | AT1G77920 | TGA7 | Transcription factor TGA7 |
| XP_030485434.1 | AT1G22040 | | F-box/kelch-repeat protein At1g22040 |
| XP_030484730.1 | AT1G27660 | BHLH110 | Transcription factor bHLH110 |
| XP_030484853.1 | AT1G75410 | BLH3 | BEL1-like homeodomain protein 3 |
| XP_030484998.1 | AT2G02040 | NPF8.3 | Protein NRT1/PTR FAMILY 8.3 |
| XP_030485240.1 | AT5G42520 | BPC6 | BPC6 |
| XP_030485687.1 | AT1G56130 | | Probable LRR receptor-like serine |
| XP_030484659.1 | AT3G50940 | | AAA-ATPase |
| XP_030484898.1 | AT1G19715 | JAL3 | Jacalin-related lectin 3 |
| XP_030485302.1 | AT1G75730 | | At1g75730 |
| XP_030485637.1 | AT5G47840 | AMK2 | Adenylate kinase 2, chloroplastic |
| XP_030485402.1 | AT1G22140 | | At1g22140/F2E2_13 |
| XP_030485289.1 | AT5G42560 | HVA22I | HVA22-like protein i |
| XP_030485290.1 | AT5G42560 | HVA22I | HVA22-like protein i |
| XP_030479711.1 | AT1G35420 | | Alpha/beta-Hydrolases superfamily protein |
| XP_030485640.1 | AT5G47850 | CCR4 | Serine/threonine-protein kinase-like protein CCR4 |
| XP_030485552.1 | AT5G02080 | | DNA/pantothenate metabolism flavoprotein |
| XP_030484506.1 | AT1G75280 | | Isoflavone reductase homolog P3 |
| XP_030485626.1 | AT4G08330 | | Uncharacterized protein At4g08330, chloroplastic |
| XP_030485106.1 | AT3G54350 | emb1967 | Forkhead-associated (FHA) domain-containing protein |
| XP_030485359.1 | AT1G36160 | ACC1 | Acetyl-CoA carboxylase 1 |
| XP_030485277.1 | AT1G19860 | | Zinc finger C-x8-C-x5-C-x3-H type family protein |
| XP_030485669.1 | AT5G44250 | | At5g44250 |
| XP_030485169.1 | AT1G75620 | | F10A5.18 |
| XP_030485180.1 | AT1G19900 | | At1g19900/F6F9_4 |
| XP_030485088.1 | AT1G75520 | SRS5 | Protein SHI RELATED SEQUENCE 5 |
| XP_030485685.1 | AT4G08240 | | At4g08240 |
| XP_030485577.1 | AT5G11320 | YUC4 | YUC4 |
| XP_030485686.1 | AT4G08240 | | At4g08240 |
| XP_030486622.1 | AT1G72100 | | Late embryogenesis abundant domain-containing protein |
| XP_030485656.1 | AT1G35460 | BHLH80 | Transcription factor bHLH80 |
| XP_030485638.1 | AT1G72180 | CEPR2 | Receptor protein-tyrosine kinase CEPR2 |
| XP_030485611.1 | AT5G25620 | YUC6 | Flavin-binding monooxygenase family protein |
| XP_030485280.1 | AT1G19850 | ARF5 | Auxin response factor |
| XP_030485288.1 | AT1G75690 | LQY1 | LQY1 |
| XP_030485654.1 | AT1G22540 | NPF5.10 | Protein NRT1/PTR FAMILY 5.10 |
| XP_030486599.1 | AT1G14340 | | RNA-binding (RRM/RBD/RNP motifs) family protein |
| XP_030485454.1 | AT1G22060 | | FBD, F-box and Leucine Rich Repeat domains containing protein |
| XP_030485399.1 | AT1G22140 | | At1g22140/F2E2_13 |
| XP_030485048.1 | AT1G75520 | SRS5 | Protein SHI RELATED SEQUENCE 5 |
| XP_030485628.1 | AT3G05270 | FPP3 | Filament-like plant protein 3 |

Based on the refined candidate list, the inventors searched for genes that may encode proteins involved in the stress response or those that could play a role in floral development from an annotated gene list for this region of NC_044370.1 from the Cannabis sativa CS10 genome. Upon inspection of this genomic region of the QTL between 102037098 to 104628858 and NCBI BLAST analysis of putative candidates they identified seven candidate genes with NCBI references: LOC115701253, LOC115701243, LOC115703213, LOC115701709, LOC115701693, LOC115703227, LOC115701761 (Table 7) that functionally make sense in the context of the flower density trait.

LOC115701253 and LOC115701243 encode proteins both with homology to Arabidopsis thaliana BLH3. BLH3 is a member of the BEL1-like family in Arabidopsis consisting of 13 members. Members of this protein family have been found to play roles in the transition from vegetative to reproductive phase and may have roles in meristem maintenance. The proximity of these genes to the most significantly associated SNP, “common_563” and their role in plant development suggest they may be involved in regulating the flower density trait.

LOC115703213 encodes a protein with homology to Arabidopsis thaliana Cytokinin dehydrogenase (CKX). CKX catalyzes the irreversible deactivation of cytokinin. Cytokinin's are a major plant hormone that play essential roles in plant growth and morphogenesis, particularly at the level of cell division and expansion. The regulation of the cytokinin dehydrogenase can impact cytokinin levels having an impact at various stages of plant development. In Arabidopsis, which has a raceme type inflorescence architecture, overexpression of CKX decreased cytokine levels and resulted in a plant that produced very few flowers. Cytokinin increases can lead to plants with larger inflorescence meristems and an increase in flower number. In wheat, barley, and rice the downregulation of CKX can improve important agronomic characteristics like yield, grain number, flower number, and grain weight. LOC115703213 is in close proximity, 123118 bp away, to the most significantly associated SNP, “common 563” making it a likely candidate. The downregulation of LOC115703213 and the decreased protein expression of CKX may increase cytokinin levels in Cannabis stimulating higher flower density.

Finally, LOC115701693 encodes a protein with homology to Arabidopsis thaliana ARF5, while LOC115703227 and LOC115701761 encode auxin-responsive factor-like proteins. Auxin may play a role in the regulation of flower density by regulating genes that specify the site of flower initiation thereby regulating flower patterning.

TABLE 7
Gene list of candidate genes identified on chromosome NC_044370.1.
The gene ID is provided with reference to the publicly available
CS10 genome as updated in April 2020 and accessed in February 2022.

| Start | End | | | |
|---|---|---|---|---|
| Position | Position | Gene ID | Protein ID | Description |
| 102987466 | 102990135 | LOC115701253 | XP_030484864.1 | BEL1-like |
| 102992742 | 102996472 | LOC115701243 | XP_030484853.1 | BEL1-like |
| 103013761 | 103019751 | LOC115703213 | XP_030486587.1 | Cytokinin dehydrogenase |
| 103525077 | 103526851 | LOC115701693 | XP_030485279.1 | auxin-responsive factor |
| | | | | AUX/IAA-related |
| 103624695 | 103625617 | LOC115703227 | XP_030486601.1 | auxin-responsive factor |
| | | | | AUX/IAA-like protein |
| 103704381 | 103705454 | LOC115701761 | XP_030485284.1 | auxin-responsive factor |
| | | | | AUX/IAA-like protein |

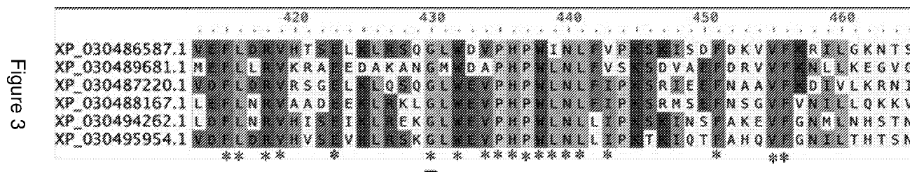

The candidate genes in Table 7 were inspected for the effect of the identified SNPs in Table 5. The inventors found that a SNP from Table 5 at position 103015433 in the gene LOC115703213 encoding a cytokinin dehydrogenase XP_030486587.1 (termed “pos_1093_A”) resulted in a radical amino acid replacement of a glycine to a glutamic acid, G365 (GGA) to E365 (GAA). Based on the Schmülling et al. 2003 paper the inventors identified 6 CKX genes in the genome of CS10 based on the amino acid consensus sequence of CKX proteins. This finding based on comparison to the consensus sequence supports the identification of XP_030486587.1 as well as the 5 other CKX proteins in the CS10 genome, listed in FIG. 3, as a functional cytokinin oxidase. All CKX proteins identified, including the CKX in CS10 were found to encode proteins with a conserved glycine that aligns with G365 in XP_030486587.1 (FIG. 3). The glycine at this position is highly conserved in all CKX proteins including in diverse species—Zea mays, Oryza sativa, Dendrobium sp., and Nostoc sp.

A functional CKX would catalyse the deactivation of cytokinin to adenine and 3-methylbut-2-enal (or another aldehyde in case of different substrate). Surprisingly, the inventors found that in the extensive high resin Cannabis varieties tested in their collection with dense flower structure, for example 20 000 070 0000, were homozygous for the allele that resulted in G365 to be substituted by glutamic acid, E365. The loss of function mutation caused by G365>E365 is predicted to lead to the loss of cytokinin oxidase activity during flower development thereby leading to increased cytokinin levels stimulating the flower density phenotype observed.

Coding sequence of XP_030486587.1 in CS10 and in GID: 20 000 070 0000. Position of the SNP leading to the G365>E365 change is bold and underlined:

>cs10_XP_030486587.1
(SEQ ID NO: 43)
ATGGAACTAACGGATGTTCTCCGGCTAGCAATTGACGGCCAGCTAAGCCT
TGACCAAGCTGACGTGGAAATGGCTTCTAAGGATTTTGGTTTAATGAAAC
GAGCGAAGCCGTTAGCCGTGTTGCACCCGGCGTCGGCTGAGGACGTGGCA
AGGGTAGTGAGAGCGGCTTACAGGTCGAGTTGGGGGCTGACTGTTTCGGC
AAGAGGAGAAGGGCATTCCATAAACGGTCAAGCCCAGACGAAGAACGGGA
TTGTGATTGCAATGAGCAGATCGTGTTCGTGGGGAATGAAGAAGAAGAAG
TCGAAGTCGAAATCGAAGGAGCAGGAGTCGTTGGATCAGCGGCCGAGACC
TCGAGTTTGTGTAGAAGAGATGTTCGTGGACGTTTGGGGTGGGGAGCTAT
GGATACAGGTGTTAATGGATACCCTAATGCATGGGCTGGCTCCCAAGTCA
TGGACTGATTACTTGTACTTATCAGTTGGAGGAACCCTTTCCAATGCTGG
AATTAGCGGTCAAACCTTTAATCATGGTCCTCAAATCAGTAATGTTCATG
AACTCGACGTCGTTACAGGCAAAGGTGAGCTGATGACTTGTTCAGAAGAG
AAAAACTCAGAGCTCTTCTACGCAGTTCTAGGTGGTCTAGGCCAATTTGG
AATTATAACTAGGGCAAGAATTGCTCTTGAACCAGCTCCTGAAAGGGTGA
AGTGGATGAGAGTACTGTATTCCGATTTCATGGCATTCACCAAAGACCAA
GAGTTTCTCATCTCTTTGCATGGACAACCTAGTCCCCAAAAATTCGACTA
TGTGGAGGGTTTTGTTATTGTAGATGACAGCCTTATTAACAGTTGGAGGT
CTTCTTTTTTGTCACCACTAAATCCAATCAAACTTTCTTCCGTTAATCCT
GACGGAGGTGTGTTATATTGCTTGGAGATAACCAAAAACTATGCTGAATC
CGATGCTCACACCGTTGATGAGGATATTGAGGCATTGTCAAAGAAACTGA
AGTTTATACCTAACTTGGTATTCAAAACGGATGTTCCGTACGTGGAATTC
TTGGACCGAGTCCACACATCCGAGTTGAAACTCAGGTCCCAGGGATTGTG
GGACGTACCCCACCCTTGGATCAACCTTTTTGTCCCCAAGTCAAAGATTT
CTGACTTTGATAAGGTTGTGTTCAAAAGAATTTTGGGTAAAAACACCAGT
GGGCCCATTCTTATCTACCCCATGAACAAACACAAATGGAACGAAAGGAG
CTCGGTGGTTACACCAGATGAGGAGGTGTTTTACGTGGTGGGATTGTTAA
GATCGGCAGCATCATCAGGCTCAGCCAATAATGATAGTATTAATGATGAT
ATTGATGAGACACAAAGTGTGGAGTACTTGAGCCAACAGAATGATGATAT
AGTGAAATACTGTGGTGAAGCTGGGATCATGGTCAAGAAATACCTACCCC
ACTTCGAAACTCAGGAGGAGTGGATGGACCACTACGGACAAAAGTGGGAT
CACTTTCTCAAACTCAAGAACAAGTTCGACCCTCGTCGCGTATTAGCCAC
TGGCCAGCGTATATTCACCACTAATATGAACAAGAAAACAAAAAAATAT
>GID: 20 000 070 0000_XP_030486587.1
(SEQ ID NO: 44)
ATGGAACTAACAGATGTTCTCCGGCTAGCAATTGACGGCCAGCTAAGCCT
TGACCAAGCTGACGTGGAAATGGCTTCTAAGGATTTTGGTTTAATGAAAC
GAGCGAAGCCGTTAGCCGTGTTGCACCCGGCGTCGGCTGAGGACGTGGCA
AGGGTAGTGAGAGCGGCTTACAGGTCGAGTTGGGGGCTGACTGTTTCGGC
AAGAGGAGAAGGGCATTCCATAAACGGTCAAGCCCAGACGAAGAACGGGA
TTGTTATTGCAATGAGCAGATCGTGTTCGTGGGGAATGAAGAAGAAGAAG
TCGAAGTCGAAATCGAAGGAGCAGGAGTCGTTGGATCAGCGGCCGAGACC
TCGAGTTTGTGTAGAAGAGATGTTTGTGGACGTTTGGGGTGGGGAGCTAT
GGATACAGGTGTTAATGGATACCCTAATGCATGGGCTGGCTCCCAAGTCA
TGGACTGATTACTTATACTTATCAGTTGGAGGAACCCTTTCCAATGCTGG
AATTAGCGGCCAAACCTTTAATCATGGTCCTCAAATCAGTAATGTTCATG
AACTCGACGTTGTTACAGGCAAAGGTGAGCTGATGACTTGTTCAGAAGAG
AAAAACTCAGAGCTCTTCTACGCAGTTCTAGGTGGTCTAGGCCAATTTGG
AATTATAACTAGGGCAAGAATTGCTCTTGAACCAGCTCCTGAAAGGGTGA
AGTGGATGAGAGTACTGTATTCCGATTTCATGGCATTCACCAAAGACCAA
GAGTTTCTCATCTCTTTGCATGGACAACCTAGTCCCCAAAAATTCGACTA
TGTGGAGGGTTTTGTTATTGTAGATGACAGCCTTATTAACAGTTGGAGGT
CTTCTTTTTTGTCACCACTAAATCCAATCAAACTTTCTTCCGTTAATCCT
GACGGAGGTGTGCTATATTGCTTGGAGATAACCAAAAACTATGCTGAATC
CGATGCTCACACCGTTGATGAGGATATTGAGGCATTGTTAAAGAAACTGA
AGTTTATACCTAACTTGGTATTCAAAACGGATGTTCCGTACGTGGAATTC
TTGGACCGAGTCCACACATCCGAGTTGAAACTCAGGTCCCAGGAATTGTG
GGACGTACCCCACCCATGGATCAACCTTTTTGTCCCCAAGTCAAAGATTT
CTGACTTTGATAAGGTTGTGTTCAAAAGAATTTTGGGTAAAAACACCAGT
GGGCCCATTCTTATCTACCCCATGAACAAACACAAATGGAACGAAAGGAG
CTCGGTGGTTACACCAGATGAGGAGGTGTTTTACGTGGTGGGATTGTTAA
GATCGGCAGCATCATCAGGCTCAGCCAATAATGATAGTATTAATGATGAT
ATTGATGAGACACAAAGTGTGGAGTACTTGAGCCAACAGAATGATGATAT
AGTGAAATACTGTGGTGAAGCTGGGATCATGGTCAAGAAATACCTACCCC
ACTTCGGAACTCAGGAGGAGTGGATGGACCACTACGGACAAAAGTGGGAT
CACTTTCTCAAACTCAAGAACAAGTTCGACCCTCGTCGCGTTTTAGCCAC
TGGCCAGCGTATATTCACCACTAATATGAACAAGAAAACAAAAAATTAT

### Example 3

Gene Identification Based on Correlation with Significant SNP “Common_563”

Preliminary analysis set out in Example 2 resulted in the identification of SNPs in genes within the region of the flower density QTL that will result in amino acid changes to expressed proteins (Table 5). Additionally, gene candidates associated with those SNPs were listed in Table 6. The inventors further filtered the SNPs identified in Table 5 by testing if the variant position was correlated with the significant SNP “common_563” from the results of the GWA marker panel in Example 1; and assigned an FDR score based on the correlation. Only proteins with significant FDR score were considered. The inventors then evaluated the remaining SNPs and associated proteins with homologs in Arabidopsis as candidates, resulting in a surprising additional candidate gene, LOC115702276 (Table 6), where two SNPs were found (Table 5). LOC115702276 encodes the protein with ID, XP_030485585, a transcriptional corepressor LEUNIG isoform X1-X4. XP_030485585 contains three known domains: 1) a N-terminal LisH domain that mediates dimer- and trimerization and is a hallmark if transcriptional repressors; 2) a C-terminal WD40 domain, a domain known to coordinate interactions with other proteins; and 3) a coiled coil domain. The protein candidate displays structural features similar to that of plant Gro/Tup1 co-repressors which include LEUNIG, TOPLESS, and WUSCHEL-INTERACTING PROTEINS in Arabidopsis. These co-repressors are implicated in floral and embryo developmental processes and in stem cell maintenance at the shoot apex.

Leunig has been identified be a regulator of AGAMOUS, where in Arabidopsis, mutations in LEUNIG cause unregulated AGAMOUS mRNA expression leading to homeotic transformations of floral organ identity as well as loss of floral organs.

The inventors focused on the gene LOC115702276 and its protein model of XP_030485585 and determined that the two SNPs detected, 104261554 (A/G) and 104263067 (T/C), were the only SNPs present in this gene in the genome collection tested, the SNPs are named “Leun229” and “Leun424”, respectively. The SNPs are tightly linked, where in all lines when 104261554 is (A) then is 104263067 (T), and in the alternative as well. The SNP at 104261554 underlies an amino acid change at position 229 Threonine (ACA) to Alanine (GCA), the SNP at 104263067 underlies an amino acid change at position 424 Leucine (CTG) to Proline (CCG). The inventors sought to understand if there were any secondary structure features disrupted particularly by 424 L>P by submitting the reference and alternative protein sequences to PSIPRED, a secondary structure prediction program (http://bioinf.cs.ucl.ac.uk/psipred). Surprisingly, the inventors found that the 424 L>P shifted the position of an alpha-helix formed at this position while 229 T>A caused no clear structural affect. Disruption to the secondary structure of a protein can disrupt or slightly modulate the activity or binding to other proteins or to DNA.

The inventors then looked to validate the finding that 104261554 (G/A) and 104263067 (T/C) underlie variation in flower density by comparing the genotype and phenotype of 15 well-characterized Cannabis high resin-type and hemp-type genotypes in their collection, named PG1-PG15. The inventors confirmed that when the genotype of the SNP is 104261554 (A) and 104263067 (T), 229 Thr and 424 Leu, the plants are THC varieties with dense flowers (Table 8). Alternatively, the inventors found that when the genotype of the SNP is 104261554 (G) and 104263067 (C), 229 Ala and 424 Pro, the plants are hemp varieties with a low flower density (Table 8). The inventors propose that targeted gene editing of the identified SNPs in LOC115702276 can be used to manipulate flower density in Cannabis.

The inventors identify that the SNPs identified in LOC115702276 may be used as genetic markers for the discrimination of high vs low density flowers at the genetic level. In order to validate the effectiveness of the SNPs at 104261554 (G/A) and 104263067 (T/C), for use in marker assisted selection, a collection of 67 diverse Cannabis varieties were grown to harvest in the field in a field trial in Niederwil, Switzerland in 2021 and 2022. The inventors chose high resin-type THC, high resin-type CBD and hemp-type varieties for the trial. Flowers were harvested and air dried, scoring was conducted by visual inspection and plants were determined to be high, medium or low density. The 67 Cannabis varieties tested comprised part of a sequence proprietary pangenome study, the inventors extracted the genotype information for the SNPs at 104261554 (G/A) and 104263067 (T/C) and compared this to the phenotypic information derived from the trial. The inventors found that both SNPs at 104261554 (G/A) and 104263067 (T/C) had a 100% accuracy in selecting the correct phenotype based on genotype information.

Table 8. Genotype and phenotype table of 15 sequenced and assembled lines (PG1 to PG15), which were genotyped for SNPs on chromosome NC_044370.1 at position 104261554 and at 104263067 (with reference to the CS10 genome). Phenotypes of the plants are given with respect to the amino acid changes found as the result of the SNPs at 229 and 424 (HD THC-high flower density THC or as LD Hemp-low flower density hemp).

| | 104261554 | 104263067 | 229Ala/424Pro | 229Thr/424Leu |
|---|---|---|---|---|
| PG1 | A/A | T/T | | HD [symbol] THC |
| PG2 | A/A | T/T | | HD [symbol] THC |
| PG3 | A/G | T/C | LD [symbol] Hemp | |
| PG4 | G/G | C/C | LD [symbol] Hemp | |
| PG5 | A/A | T/T | | HD [symbol] THC |
| PG6 | G/G | C/C | LD [symbol] Hemp | |
| PG7 | A/A | T/T | | HD [symbol] THC |
| PG8 | A/A | T/T | | HD [symbol] THC |
| PG9 | A/A | T/T | | HD [symbol] THC |
| PG10 | G/G | C/C | LD [symbol] Hemp | |
| PG11 | G/G | C/C | LD [symbol] Hemp | |
| PG12 | G/G | C/C | LD [symbol] Hemp | |
| PG13 | G/G | C/C | LD [symbol] Hemp | |
| PG14 | G/G | C/C | LD [symbol] Hemp | |
| PG15 | G/G | C/C | LD [symbol] Hemp | |

Coding sequence of gene LOC115702276 with the SNPs at positions 104261554 (G/A) and 104263067 (T/C) indicated in square brackets and shown underlined and in bold (SEQ ID NO:45). The first nucleotide indicated in square brackets at each position is the nucleotide of CS10 reference genome sequence (high flower density) and the second nucleotide indicated in square brackets at each position is the nucleotide of the PG3 line (low flower density):

ATGGCTCAGACCAACTGGGAAGCAGATAAAATGTTAGATGTGTACATCCA
CGATTATTTAGTAAAGAGGGATTTAAAGGCTTCTGCTCAAGCATTCCAAG
CTGAAGGGAAAGTGTCATCGGATCCTGTTGCTATTGATGCTCCTGGAGGC
TTTCTCTTTGAATGGTGGTCTGTATTCTGGGATATATTTATAGCTAGAAC
CAATGAGAAGCATTCAGAAGTTGCTGCGTCTTATATTGAGACACAGTTAA
TTAAAGCAAGGGAACAACAGCAGCAGCAGCAGCAGTTCCAACAACAACAA
CCACAGCAACCACAGCACCAACAACAACAGCAGCAGCACATCCAAATGCA
ACAACTTCTGTTGCAGAGGCATGCTCAGCAGCAACAGCAACAGCAACAGC
AACAGCAACAACAGCAGCAGCAGGCACCACCACAACAGCAGCGAAGAGAA
GGGAGCCACATTTTAAATGGTACTACTAATGGACTTGTTGGAAGTGATCC
GCTCATGCGACAAAATCCAGGAACAGCTAATGCCATGGCTACAAAGATGT
ATGAGGAGCGATTAAAGCTGCCTTCTCAAAGAGATCCTTTAGATGATGCA
GCTATGAAGCAGCAAAGATTTGGTGAGAGTGTGGGCCAACTTTTGGATCC
AACAAGTCAGACCTCCATATTAAAGTCACCTGCA[A/G]CATCCAGCCAG
CCATCAGGGCAAGTATTGCATGGTTCAGCTGGAGCGATGTCTCCTCAAGT
TCAAGCTCGAAATCAACAATTGCCAGGGTCCACCCCGGACATAAAGCCTG
AAACTAATCCAGTTTTGAACCCCCGAGGTGCTGGTCAAGAAGGATCATTA
GTAGGAATGCCCGGGTCAAATCAAGGAGGAAACAATTTGACTTTAAAAGG
GTGGCCTCTGACAGGTCTGGATCATATTCGCACTGGGTTTCTCCAGCAAC
AAAAGCCTTTTATGCAGGCTCCTCAGCCCTTTCATCAACTTCAGATGTTG
ACACCACAACACCAACTTATGCTTGCACAACAAAATTTGTCCTCACCATC
TGCTAGTGATGAAAATAGAAGACTTAGAATGCTTTTGAGTAACCGAAATT
TGGGTCTTGTAAAGGATGGCTTTTCGAGTTCTGTTGGAGATGTGGTTCCC
AATGTTGGATCCCAACTTCAAGGGAGTGGGGGTCATGTTTTGCCTCGTGG
AGATACGGATATGCTGATTAAGTTAAAAATGGCTCAACTACAGCAGCAGC
AACAGCAACAACAGAACAGTACAC[T/C]GTCACAGCAGCTACAGCAACA
TTCAAATCAGCAGTCACAGAGTTCCAATCACAATCCACACCAAGATAAGA
TGGGTGGTGCTGGCAGTGTGACCATGGACGGTAGCATATCAAACTCCTTT
CGAGGAAATGACCAGGGTTCAAAAAGCCAGACTGGTAGAAAGCGAAAACA
GCCAGTGTCATCTTCGGGTCCTGCAAATAGCTCAGGAACGGCAAATACAG
CTGGACCTTCACCAAGCTCAGCTCCCTCAACACCTTCAACTCACACACCT
GGGGATGTGATCTCAATGCCTGCATTGACCCATAGTGGTAGTTCTTCAAA
GCCTTTCATATTTGCAGCTGATGGTACTGGTACTCTTACATCACCATCAA
ATCAGTTGTGGGATGATAAAGATCTTGAATTGCAGGCTGATATGGATCGA
TTTGTAGATGATGGATCCCTCGAGGACAATGTGGAGTCTTTCTTATCCCA
TGATGATGCAGACCCCAGAGATGCTGCTGGTCGTTGTATGGATGTTAGCA
AAGGGTTTACATTTGCAGAAGTAAATTCTGTTAGAGCAAGCACGAGCAAA
GTTATATGTTGTCACTTCTCATCGGATGGAAAACTGCTTGCTAGTGGTGG
CCATGATAAAAAGGCTGTATTATGGTACACGGATTCCTTAAAGTCTAAAA
CTTCACTTGAGGAACATTCAGCATTGATTACTGATGTTCGTTTTAGTCCA
AGCATGTCACGGCTTGCTACGTCCTCATTTGATAAAACTGTTAGGGTTTG
GGATGCTGACAATCCCGGTTATTCACTACGCACCTTTATGGGACATTCCA
ACACTGTAATGTCATTAGACTTCCACCCAAATAAAGAAGATCTTCTCAGC
TCTTGTGACAGCGATGGTGAGATACGATATTGGAGTATTAACAATGGCAG
TTGTGCTAGAGTGTTCAAGGGTGGTACGGCACAGATGAGATTCCAACCCC
GTTTTGGGAGGTACCTGGCTGCAGCTGCAGATAACCTTGTATCTATACTG
GATGTGGAGACTCAAGTTTGTCGGAATTCACTACAGGGGCATACTAAGCC
AGTCCATTCTGTGTGCTGGGATCCTTCGGGTGAGTTACTTGCATCGGTGA
GTGAGGACTCCGTCAGAGTCTGGTCGCTTGGGTCAGGAAATGAAGGGGAA
TGTGTTCACGAGTTGAGCTGCAGCGGAAATAAATTCCATTCCTGTGTTTT
CCATCCTACTTATCCTTCACTGTTAGTTGTAGGCTGTTACCAGTCGTTGG
AGCTCTGGAACATGATGGAAAACAAGACAATGACAATATCAGCTCATGAA
GGACTTATTGCAGCCTTGGCCGTGTCCCCTCTCACAGGTTTGGTAGCTTC
AGCCAGTCATGATAAGTTTGTCAAGCTTTGGAAGTGAGACCAGTTCCTGC
CCCCCTCCTACTAGTTTAACCATACAGGATTAGCTTCTTCTCAGGATCGA
TTAAATTGGATGTAGTAGTTTGTTTCTGCCTCGGTTATTCGATTTTTTTT
CTTCTTAACGTCCTATGTAACTTCTGAACTTTGACCAAGTAAAATATTAT
TTCTCTTGTTGTATTTGTATCCATGCTTTTGCATACCTGTATTTGGTTGC
TCAATTAATTTTGAAAAACCTGAACTTTGCTTCATCCTCCAGTTTCTA

1. A method for characterizing a Cannabis spp. plant with respect to a flower density trait, the method comprising the steps of:
(i) genotyping at least one plant with respect to a flower density QTL by detecting: (a) one or more polymorphisms associated with the flower density trait as defined in Table 2 or 3; and/or (b) a polymorphism causal for the flower density trait selected from a A/G SNP at position 685 of SEQ ID NO:45 and a T/C SNP at position 1271 of SEQ ID NO:45, or both; and
(ii) characterizing the plant with respect to the flower density QTL as having an increased flower density QTL, a decreased flower density QTL or an intermediate flower density QTL based on the genotype at the polymorphism.

2. The method of claim 1, wherein the polymorphism is selected from the consisting of group “common_563”, “GBScompat_rare_14”, “common 573”, “GBScompat_common_102”, “rare_66”, “common_583”, as defined in Table 2 or 3, the A/G SNP at position 685 of SEQ ID NO:45, the T/C SNP at position 1271 of SEQ ID NO:45, and combinations thereof.

3. (canceled)

4. (canceled)

5. The method of claim 1, wherein the genotyping is performed by PCR-based detection using molecular markers, sequencing of PCR products containing the one or more polymorphisms, targeted resequencing, whole genome sequencing, or restriction-based methods, for detecting the one or more polymorphisms.

6. The method of claim 5, wherein the molecular markers are for detecting polymorphisms at regular intervals within the flower density QTL such that recombination can be excluded or such that recombination can be quantified to estimate linkage disequilibrium between a particular polymorphism and the flower density trait, optionally wherein the molecular markers are designed based on a context sequence for the polymorphism in Table 3 or are selected from the primer pairs as defined in Table 4.

7. (canceled)

8. The method of claim 1, wherein the flower density QTL is a quantitative trait locus having a sequence that corresponds to nucleotides 102037098 to 104628858 of NC_044370.1 of the CS10 genome and is defined by one or more polymorphisms associated with flower density as defined in Table 2 or 3, or a genetic marker linked to the QTL.

9. A method of producing a Cannabis spp. plant having a flower density trait of interest, the method comprising the steps of:
(i) providing a donor parent plant having in its genome a flower density QTL characterized by: (a) one or more polymorphisms associated with the flower density trait of interest as defined Table 2 or 3; and/or (b) a polymorphism causal for the flower density trait of interest selected from a A/G SNP at position 685 of SEQ ID NO:45 and a T/C SNP at position 1271 of SEQ ID NO:45, or both:
(ii) crossing the donor parent plant having the flower density QTL with at least one recipient parent plant to obtain a progeny population of Cannabis plants;
(iii) screening the progeny population of Cannabis plants for the presence of the flower density QTL; and
(iv) selecting one or more progeny plants having the flower density QTL, wherein the mature plant displays the flower density trait of interest.

10. The method of claim 9, further comprising:
(v) crossing the one or more progeny plants with the donor recipient plant: or
(vi) selfing the one or more progeny plants.

11. The method of claim 9, wherein the screening comprises genotyping at least one plant from the progeny population with respect to the flower density QTL by detecting the one or more polymorphisms associated with the flower density trait of interest as defined Table 2 or 3; and/or the polymorphism causal for the flower density trait of interest, and optionally wherein the method further comprises a step of genotyping the donor parent plant with respect to the flower density QTL by detecting the one or more polymorphisms associated with flower density trait of interest as defined Table 2 or 3; and/or the polymorphism causal for the flower density trait of interest, prior to step (i).

12. (canceled)

13. The method of claim 11, wherein the genotyping is performed by PCR-based detection using molecular markers, sequencing of PCR products containing the one or more polymorphisms, targeted resequencing, whole genome sequencing, or restriction-based methods, for detecting the one or more polymorphisms.

14. The method of claim 13, wherein the molecular markers are for detecting polymorphisms at regular intervals within the flower density QTL such that recombination can be excluded or such that recombination can be quantified to estimate linkage disequilibrium between a particular polymorphism and the flower density trait of interest, optionally wherein the molecular markers are designed based on a context sequence for the polymorphism in Table 3 or are selected from the primer pairs as defined in Table 4.

15. (canceled)

16. The method of claim 9, wherein the polymorphism is selected from the group consisting of “common_563”, “GBScompat_rare_14”, “common_573”, “GBScompat_common_102”, “rare_66”, “common_583”, as defined in Table 2 or 3, the A/G SNP at position 685 of SEQ ID NO:45, the T/C SNP at position 1271 of SEQ ID NO:45, and combinations thereof.

17. (canceled)

18. (canceled)

19. The method of claim 9, wherein the flower density QTL is an increased flower density QTL, a decreased flower density QTL, or an intermediate flower density QTL.

20. The method of claim 9, wherein the flower density QTL is a quantitative trait locus having a sequence that corresponds to nucleotides 102037098 to 104628858 of NC_044370.1 of the CS10 genome and is defined by one or more polymorphisms associated with flower density as defined in Table 2 or 3, or a genetic marker linked to the QTL.

21. A method of producing a Cannabis spp. plant comprising a flower density trait of interest, the method comprising introducing into a Cannabis spp. plant a flower density QTL:
(a) characterized by one or more polymorphisms associated with the flower density trait of interest as defined in Table 2 or 3, wherein said flower density QTL is associated with the flower density trait of interest in the plant; and/or
(b) comprising a polymorphism causal for the flower density trait of interest selected from a A/G SNP at position 685 of SEQ ID NO:45 and a T/C SNP at position 1271 of SEQ ID NO:45, or both.

22. The method of claim 21, wherein introducing the flower density QTL comprises crossing a donor parent plant having the flower density QTL with a recipient parent plant or genetically modifying the Cannabis spp. plant.

23. (canceled)

24. The method of claim 22, wherein genetically modifying the Cannabis spp. plant is by targeted mutagenesis of a nucleotide at position 685 of SEQ ID NO:45, at position 1271 of SEQ ID NO:45, or both.

25. The method of claim 21, wherein the flower density QTL is a quantitative trait locus having a sequence that corresponds to nucleotides 102037098 to 104628858 of NC_044370.1 of the CS10 genome and is defined by one or more polymorphisms associated with flower density as defined in Table 2 or 3, or a genetic marker linked to the QTL.

26. (canceled)

27. (canceled)

28. A Cannabis spp. plant comprising a flower density QTL, wherein the flower density QTL is:
(a) characterized by one or more polymorphisms associated with a flower density trait of interest as defined in Table 2 or 3, wherein said flower density QTL is associated with the flower density trait of interest in the plant; and/or
(b) comprising a polymorphism causal for the flower density trait of interest selected from a A/G SNP at position 685 of SEQ ID NO:45 and a T/C SNP at position 1271 of SEQ ID NO:45, or both.

29. (canceled)

30. (canceled)

31. (canceled)


SEQUENCE LISTING (45 sequences)

SEQ ID NO: 1 - DNA, 403 bp, Cannabis sp.
  source 1..403: mol_type = other DNA
  variation 202: allele = n = a/t
ggagtgtaaatgcctacacaaagaaaattttatgaattagataaatctttcttcacaacacaaacagttgatactaagaaatcagctaaaaggtgaaatgaaaacttgaagcaagagctacaaataattaccgtaggctgaataagaagacaagactataatgtatgctgaagctcttttgattgaataccacagaggtgangtactctttgaagctctgtatggaacatgatacctgtttttataaggtttgaaaggtttaccctcaccctgcaataaaatttccaaaatttggtccatatattaagagcaattacaaccaaatggaatggcactactgagggtagaaataacaaagcatttcaggataaaagtaaagctcaagacatatcacaatttaaga

SEQ ID NO: 2 - DNA, 403 bp, Cannabis sp.
  source 1..403: mol_type = other DNA
  variation 202: allele = n = c/a
gagtctgtgtcaaaggaaactgatgtagggaccattttggaggatatcaaatgtgccatccaggagacacatgatactctccaccagcacgcagtaagttgtgtttctgaagatgttcattgttctgatgccagatctgataggcaggtcaatccagaagatgctggcttaactgctgataaggaaatttctctttctcagncagccagggaaagtgagcagattagcaaagatttagctgctgccatttctcagatccatgattttgtaaccttcttgggcaaagaagctatggtagtccacgacactttcaccgaaagcgatggattcagcaagagaattgaagatttctctgtgacctataataaagtcatgcacagcaacctaagtttacttgattttg

SEQ ID NO: 3 - DNA, 403 bp, Cannabis sp.
  source 1..403: mol_type = other DNA
  variation 202: allele = n = g/a
ttgacaacgtcccacctttctaattgactgattttggaccactgggttagttcttggggaacaagatggaacatcaagccaagctggaattttagcagttggtaacctgcaatagtaaaagctaaattaaaagggtccaacataatattacaagagtaaacaagcaaaagcacttacggtgtacatgtatcattaactctancaccttgtgaaatggttgatgacggtccaggcaatgatcccattccttcaaattaaagaaggattcattttaatttccaaataatgatgataaaatgtgtttatcagaaagaccatatacacttaccagctttcttggagctcaaaataggtgtgttctgaaatggggtaccataaactacgttcagatcatagtcaaaac

SEQ ID NO: 4 - DNA, 403 bp, Cannabis sp.
  source 1..403: mol_type = other DNA
  variation 202: allele = n = g/a
ttcacggtttccaccccctccgccggttccagctccctgtccaccgtcacaggtaaatgcttctgcgaaagacataaattactacaagagcttgattcagcaacacggaggagacaggcaagagaatctgaaccctccacaatatggtaaccgttataatcaccaatcagcgatgagccaggaaatagttaacagccctaanccgagagagtcaaagccgaaaataatgaaaccttgcatatattttaatagctcaaggggctgccggcatggagccaattgtgcgtatcagcatgatgtatcataccagcagcgtggtagttccatgtcagaggtgcagaatgcaaagagaaccaaatttgatagagaaattagcagttaaaagttgtagttcccgtttgta

SEQ ID NO: 5 - DNA, 403 bp, Cannabis sp.
  source 1..403: mol_type = other DNA
  variation 202: allele = n = a/t
atcaagtaactgcaagttgggaatagaccagagtgaaccaggaagagcaccatcgagtttattgtcactcagaaccacaacccgcagctgagaaaggcttgaaaacaagccctttggcaatggaccctctagaccattagcactaatatcaataaccctcaagctcctcaaacccgtgaattctttaggaaatgacccagtnaaaaaattcttgctacaatttagctcaacaagtcgagaaagtttattcaactgaacaggtatagaagcggtaagactattatcagaagtacttaaaaactcaagtctcgagaggttacctaaacctgatggaatcgacccagaaaagaaatttgaggagaggtcaagagtagtaagatttcgaagtgaggtaaactcgaac

SEQ ID NO: 6 - DNA, 403 bp, Cannabis sp.
  source 1..403: mol_type = other DNA
  variation 202: allele = n = g/a
cagggaagctctgtgtacatataaattcagcaatgaggaagcagataaaattcttggaaaagcttttggccagatacactcgccttactggggtgaagaacgcaaaaaggttgttccgaaacttgaacttgtgaatgaaattttggactacttaagaaatttgaacctatcagaagatgatctcgcgaaggtgcttaaaaantttcctgaagttcttggatgtgatctagaagatgaggtaaaagtcaatgtgcaggttttggagaaggaatgggaaatcaaaggaaaaactcttcgaaaccttcttcttcgaaatccaagactattgggttacaacattgactgcaagggagattgtgcagcacaatgcacgcgatgctgggctcgtttttaagctaaatct

SEQ ID NO: 7 - DNA, 103 bp, Cannabis sp.
  source 1..103: mol_type = other DNA
  variation 52: allele = n = c/t
tttgacttggggacaaaaaggttgatccaagggtggggtacgtcccacaatncctgggacctgagtttcaactcggatgtgtggactcggtccaagaattcca

SEQ ID NO: 8 - DNA, 403 bp, Cannabis sp.
  source 1..403: mol_type = other DNA
  variation 202: allele = n = a/g
cttgttggaagtgatccgctcatgcgacaaaatccaggaacagctaatgccatggctacaaagatgtatgaggagcgattaaagctgccttctcaaagagatcctttagatgatgcagctatgaagcagcaaagatttggtgagagtgtgggccaacttttggatccaacaagtcagacctccatattaaagtcacctgcancatccagccagccatcagggcaagtattgcatggttcagctggagcgatgtctcctcaagttcaagctcgaaatcaacaattgccagggtccaccccggacataaagcctgaaactaatccagttttgaacccccgaggtgctggtcaagaaggatcattagtaggaatgcccgggtcaaatcaaggaggaaacaatttga

SEQ ID NO: 9 - DNA, 403 bp, Cannabis sp.
  source 1..403: mol_type = other DNA
  variation 202: allele = n = t/c
ttagaatgcttttgagtaaccgaaatttgggtcttgtaaaggatggcttttcgagttctgttggagatgtggttcccaatgttggatcccaacttcaagggagtgggggtcatgttttgcctcgtggagatacggatatgctgattaagttaaaaatggctcaactacagcagcagcaacagcaacaacagaacagtacacngtcacagcagctacagcaacattcaaatcagcagtcacagagttccaatcacaatccacaccaagataagatgggtggtgctggcagtgtgaccatggacggtagcatatcaaactcctttcgaggaaatgaccagggttcaaaaagccagactggtagaaagcgaaaacagccagtgtcatcttcgggtcctgcaaatag

SEQ ID NO: 10 - DNA, 23 bp, synthetic construct
  source 1..23: mol_type = other DNA
tctttcttcacaacacaaacagt

SEQ ID NO: 11 - DNA, 20 bp, synthetic construct
  source 1..20: mol_type = other DNA
ccctcagtagtgccattcca

SEQ ID NO: 12 - DNA, 24 bp, synthetic construct
  source 1..24: mol_type = other DNA
tctttcttcacaacacaaacagtt

SEQ ID NO: 13 - DNA, 20 bp, synthetic construct
  source 1..20: mol_type = other DNA
accctcagtagtgccattcc

SEQ ID NO: 14 - DNA, 20 bp, synthetic construct
  source 1..20: mol_type = other DNA
atgtgccatccaggagacac

SEQ ID NO: 15 - DNA, 20 bp, synthetic construct
  source 1..20: mol_type = other DNA
gctgaatccatcgctttcgg

SEQ ID NO: 16 - DNA, 20 bp, synthetic construct
  source 1..20: mol_type = other DNA
gtgccatccaggagacacat

SEQ ID NO: 17 - DNA, 20 bp, synthetic construct
  source 1..20: mol_type = other DNA
tgacaacgtcccacctttct

SEQ ID NO: 18 - DNA, 20 bp, synthetic construct
  source 1..20: mol_type = other DNA
gatcattgcctggaccgtca

SEQ ID NO: 19 - DNA, 22 bp, synthetic construct
  source 1..22: mol_type = other DNA
gccaagctggaattttagcagt

SEQ ID NO: 20 - DNA, 21 bp, synthetic construct
  source 1..21: mol_type = other DNA
accccatttcagaacacacct

SEQ ID NO: 21 - DNA, 19 bp, synthetic construct
  source 1..19: mol_type = other DNA
gttccagctccctgtccac

SEQ ID NO: 22 - DNA, 20 bp, synthetic construct
  source 1..20: mol_type = other DNA
gaactaccacgctgctggta

SEQ ID NO: 23 - DNA, 20 bp, synthetic construct
  source 1..20: mol_type = other DNA
ctgtccaccgtcacaggtaa

SEQ ID NO: 24 - DNA, 20 bp, synthetic construct
  source 1..20: mol_type = other DNA
tcactcagaaccacaacccg

SEQ ID NO: 25 - DNA, 20 bp, synthetic construct
  source 1..20: mol_type = other DNA
ctgggtcgattccatcaggt

SEQ ID NO: 26 - DNA, 20 bp, synthetic construct
  source 1..20: mol_type = other DNA
tctgggtcgattccatcagg

SEQ ID NO: 27 - DNA, 20 bp, synthetic construct
  source 1..20: mol_type = other DNA
ggccagatacactcgcctta

SEQ ID NO: 28 - DNA, 20 bp, synthetic construct
  source 1..20: mol_type = other DNA
tgtgctgcacaatctccctt

SEQ ID NO: 29 - DNA, 20 bp, synthetic construct
  source 1..20: mol_type = other DNA
tgcacaatctcccttgcagt

SEQ ID NO: 30 - DNA, 50 bp, synthetic construct
  source 1..50: mol_type = other DNA
gccaacttttggatccaacaagtcagacctccatattaaagtcacctgca

SEQ ID NO: 31 - DNA, 50 bp, synthetic construct
  source 1..50: mol_type = other DNA
catccagccagccatcagggtatttttttgtttttgtttttttacttttt

SEQ ID NO: 32 - DNA, 50 bp, synthetic construct
  source 1..50: mol_type = other DNA
aaaaatggctcaactacagcagcagcaacagcaacaacagaacagtacac

SEQ ID NO: 33 - DNA, 50 bp, synthetic construct
  source 1..50: mol_type = other DNA
gtcacagcagctacagcaacattcaaatcagcagtcacagagttccaatc

SEQ ID NO: 34 - DNA, 17 bp, Cannabis sp.
  source 1..17: mol_type = other DNA
tatgatgatgatgatga

SEQ ID NO: 35 - DNA, 20 bp, Cannabis sp.
  source 1..20: mol_type = other DNA
tatgatgatgatgatgatga

SEQ ID NO: 36 - DNA, 11 bp, Cannabis sp.
  source 1..11: mol_type = other DNA
cctgctgctgc

SEQ ID NO: 37 - DNA, 11 bp, Cannabis sp.
  source 1..11: mol_type = other DNA
ggtcgtcgtcg

SEQ ID NO: 38 - DNA, 14 bp, Cannabis sp.
  source 1..14: mol_type = other DNA
ggtcgtcgtcgtcg

SEQ ID NO: 39 - DNA, 15 bp, Cannabis sp.
  source 1..15: mol_type = other DNA
taccaacaccaacat

SEQ ID NO: 40 - DNA, 21 bp, Cannabis sp.
  source 1..21: mol_type = other DNA
taccaacaccaacaccaacat

SEQ ID NO: 41 - DNA, 14 bp, Cannabis sp.
  source 1..14: mol_type = other DNA
attgttgttgtagt

SEQ ID NO: 42 - DNA, 26 bp, Cannabis sp.
  source 1..26: mol_type = other DNA
attgttgttgtagttgttgttgtagt

SEQ ID NO: 43 - DNA, 1599 bp, Cannabis sativa
  source 1..1599: mol_type = other DNA
atggaactaacggatgttctccggctagcaattgacggccagctaagccttgaccaagctgacgtggaaatggcttctaaggattttggtttaatgaaacgagcgaagccgttagccgtgttgcacccggcgtcggctgaggacgtggcaagggtagtgagagcggcttacaggtcgagttgggggctgactgtttcggcaagaggagaagggcattccataaacggtcaagcccagacgaagaacgggattgtgattgcaatgagcagatcgtgttcgtggggaatgaagaagaagaagtcgaagtcgaaatcgaaggagcaggagtcgttggatcagcggccgagacctcgagtttgtgtagaagagatgttcgtggacgtttggggtggggagctatggatacaggtgttaatggataccctaatgcatgggctggctcccaagtcatggactgattacttgtacttatcagttggaggaaccctttccaatgctggaattagcggtcaaacctttaatcatggtcctcaaatcagtaatgttcatgaactcgacgtcgttacaggcaaaggtgagctgatgacttgttcagaagagaaaaactcagagctcttctacgcagttctaggtggtctaggccaatttggaattataactagggcaagaattgctcttgaaccagctcctgaaagggtgaagtggatgagagtactgtattccgatttcatggcattcaccaaagaccaagagtttctcatctctttgcatggacaacctagtccccaaaaattcgactatgtggagggttttgttattgtagatgacagccttattaacagttggaggtcttcttttttgtcaccactaaatccaatcaaactttcttccgttaatcctgacggaggtgtgttatattgcttggagataaccaaaaactatgctgaatccgatgctcacaccgttgatgaggatattgaggcattgtcaaagaaactgaagtttatacctaacttggtattcaaaacggatgttccgtacgtggaattcttggaccgagtccacacatccgagttgaaactcaggtcccagggattgtgggacgtaccccacccttggatcaacctttttgtccccaagtcaaagatttctgactttgataaggttgtgttcaaaagaattttgggtaaaaacaccagtgggcccattcttatctaccccatgaacaaacacaaatggaacgaaaggagctcggtggttacaccagatgaggaggtgttttacgtggtgggattgttaagatcggcagcatcatcaggctcagccaataatgatagtattaatgatgatattgatgagacacaaagtgtggagtacttgagccaacagaatgatgatatagtgaaatactgtggtgaagctgggatcatggtcaagaaatacctaccccacttcgaaactcaggaggagtggatggaccactacggacaaaagtgggatcactttctcaaactcaagaacaagttcgaccctcgtcgcgtattagccactggccagcgtatattcaccactaatatgaacaagaaaacaaaaaaatat

SEQ ID NO: 44 - DNA, 1599 bp, Cannabis sp.
  source 1..1599: mol_type = other DNA
atggaactaacagatgttctccggctagcaattgacggccagctaagccttgaccaagctgacgtggaaatggcttctaaggattttggtttaatgaaacgagcgaagccgttagccgtgttgcacccggcgtcggctgaggacgtggcaagggtagtgagagcggcttacaggtcgagttgggggctgactgtttcggcaagaggagaagggcattccataaacggtcaagcccagacgaagaacgggattgttattgcaatgagcagatcgtgttcgtggggaatgaagaagaagaagtcgaagtcgaaatcgaaggagcaggagtcgttggatcagcggccgagacctcgagtttgtgtagaagagatgtttgtggacgtttggggtggggagctatggatacaggtgttaatggataccctaatgcatgggctggctcccaagtcatggactgattacttatacttatcagttggaggaaccctttccaatgctggaattagcggccaaacctttaatcatggtcctcaaatcagtaatgttcatgaactcgacgttgttacaggcaaaggtgagctgatgacttgttcagaagagaaaaactcagagctcttctacgcagttctaggtggtctaggccaatttggaattataactagggcaagaattgctcttgaaccagctcctgaaagggtgaagtggatgagagtactgtattccgatttcatggcattcaccaaagaccaagagtttctcatctctttgcatggacaacctagtccccaaaaattcgactatgtggagggttttgttattgtagatgacagccttattaacagttggaggtcttcttttttgtcaccactaaatccaatcaaactttcttccgttaatcctgacggaggtgtgctatattgcttggagataaccaaaaactatgctgaatccgatgctcacaccgttgatgaggatattgaggcattgttaaagaaactgaagtttatacctaacttggtattcaaaacggatgttccgtacgtggaattcttggaccgagtccacacatccgagttgaaactcaggtcccaggaattgtgggacgtaccccacccatggatcaacctttttgtccccaagtcaaagatttctgactttgataaggttgtgttcaaaagaattttgggtaaaaacaccagtgggcccattcttatctaccccatgaacaaacacaaatggaacgaaaggagctcggtggttacaccagatgaggaggtgttttacgtggtgggattgttaagatcggcagcatcatcaggctcagccaataatgatagtattaatgatgatattgatgagacacaaagtgtggagtacttgagccaacagaatgatgatatagtgaaatactgtggtgaagctgggatcatggtcaagaaatacctaccccacttcggaactcaggaggagtggatggaccactacggacaaaagtgggatcactttctcaaactcaagaacaagttcgaccctcgtcgcgttttagccactggccagcgtatattcaccactaatatgaacaagaaaacaaaaaattat

SEQ ID NO: 45 - DNA, 2940 bp, Cannabis sp.
  source 1..2940: mol_type = other DNA
  variation 685: allele = n = a/g
  variation 1271: allele = n = t/c
atggctcagaccaactgggaagcagataaaatgttagatgtgtacatccacgattatttagtaaagagggatttaaaggcttctgctcaagcattccaagctgaagggaaagtgtcatcggatcctgttgctattgatgctcctggaggctttctctttgaatggtggtctgtattctgggatatatttatagctagaaccaatgagaagcattcagaagttgctgcgtcttatattgagacacagttaattaaagcaagggaacaacagcagcagcagcagcagttccaacaacaacaaccacagcaaccacagcaccaacaacaacagcagcagcacatccaaatgcaacaacttctgttgcagaggcatgctcagcagcaacagcaacagcaacagcaacagcaacaacagcagcagcaggcaccaccacaacagcagcgaagagaagggagccacattttaaatggtactactaatggacttgttggaagtgatccgctcatgcgacaaaatccaggaacagctaatgccatggctacaaagatgtatgaggagcgattaaagctgccttctcaaagagatcctttagatgatgcagctatgaagcagcaaagatttggtgagagtgtgggccaacttttggatccaacaagtcagacctccatattaaagtcacctgcancatccagccagccatcagggcaagtattgcatggttcagctggagcgatgtctcctcaagttcaagctcgaaatcaacaattgccagggtccaccccggacataaagcctgaaactaatccagttttgaacccccgaggtgctggtcaagaaggatcattagtaggaatgcccgggtcaaatcaaggaggaaacaatttgactttaaaagggtggcctctgacaggtctggatcatattcgcactgggtttctccagcaacaaaagccttttatgcaggctcctcagccctttcatcaacttcagatgttgacaccacaacaccaacttatgcttgcacaacaaaatttgtcctcaccatctgctagtgatgaaaatagaagacttagaatgcttttgagtaaccgaaatttgggtcttgtaaaggatggcttttcgagttctgttggagatgtggttcccaatgttggatcccaacttcaagggagtgggggtcatgttttgcctcgtggagatacggatatgctgattaagttaaaaatggctcaactacagcagcagcaacagcaacaacagaacagtacacngtcacagcagctacagcaacattcaaatcagcagtcacagagttccaatcacaatccacaccaagataagatgggtggtgctggcagtgtgaccatggacggtagcatatcaaactcctttcgaggaaatgaccagggttcaaaaagccagactggtagaaagcgaaaacagccagtgtcatcttcgggtcctgcaaatagctcaggaacggcaaatacagctggaccttcaccaagctcagctccctcaacaccttcaactcacacacctggggatgtgatctcaatgcctgcattgacccatagtggtagttcttcaaagcctttcatatttgcagctgatggtactggtactcttacatcaccatcaaatcagttgtgggatgataaagatcttgaattgcaggctgatatggatcgatttgtagatgatggatccctcgaggacaatgtggagtctttcttatcccatgatgatgcagaccccagagatgctgctggtcgttgtatggatgttagcaaagggtttacatttgcagaagtaaattctgttagagcaagcacgagcaaagttatatgttgtcacttctcatcggatggaaaactgcttgctagtggtggccatgataaaaaggctgtattatggtacacggattccttaaagtctaaaacttcacttgaggaacattcagcattgattactgatgttcgttttagtccaagcatgtcacggcttgctacgtcctcatttgataaaactgttagggtttgggatgctgacaatcccggttattcactacgcacctttatgggacattccaacactgtaatgtcattagacttccacccaaataaagaagatcttctcagctcttgtgacagcgatggtgagatacgatattggagtattaacaatggcagttgtgctagagtgttcaagggtggtacggcacagatgagattccaaccccgttttgggaggtacctggctgcagctgcagataaccttgtatctatactggatgtggagactcaagtttgtcggaattcactacaggggcatactaagccagtccattctgtgtgctgggatccttcgggtgagttacttgcatcggtgagtgaggactccgtcagagtctggtcgcttgggtcaggaaatgaaggggaatgtgttcacgagttgagctgcagcggaaataaattccattcctgtgttttccatcctacttatccttcactgttagttgtaggctgttaccagtcgttggagctctggaacatgatggaaaacaagacaatgacaatatcagctcatgaaggacttattgcagccttggccgtgtcccctctcacaggtttggtagcttcagccagtcatgataagtttgtcaagctttggaagtgagaccagttcctgcccccctcctactagtttaaccatacaggattagcttcttctcaggatcgattaaattggatgtagtagtttgtttctgcctcggttattcgattttttttcttcttaacgtcctatgtaacttctgaactttgaccaagtaaaatattatttctcttgttgtatttgtatccatgcttttgcatacctgtatttggttgctcaattaattttgaaaaacctgaactttgcttcatcctccagtttcta